(19) United States
(12) Patent Application Publication
(10) Pub. No.: US 2024/0093259 A1
(43) Pub. Date: Mar. 21, 2024

(54) METHODS TO STABILIZE MAMMALIAN CELLS

(71) Applicant: THE REGENTS OF THE UNIVERSITY OF CALIFORNIA, Oakland, CA (US)

(72) Inventors: Nathan E. Lewis, San Diego, CA (US); Philipp Spahn, San Diego, CA (US); Shangzhong Li, Cambridge, MA (US); Hooman Hefzi, Berkeley, CA (US); Isaac Shamie, La Jolla, CA (US)

(21) Appl. No.: 17/767,844

(22) PCT Filed: Oct. 9, 2020
(86) PCT No.: PCT/EP2020/078435

§ 371 Date: Apr. 8, 2022

Related U.S. Application Data
(60) Provisional application No. 62/913,324, filed on Oct. 10, 2019.

(51) Int. Cl. C12P 21/00; C12N 15/86; C12Q 1/6897

(52) U.S. Cl. CPC C12P 21/00; C12N 15/86; C12Q 1/6897; C12N 2740/15043

(57) ABSTRACT

The invention provides gene targets whose restoration leads to genome stabilization in host cells, such as Chinese Hamster Ovary (CHO) cells. Many DNA repair genes are mutated in CHO cells which compromises their ability to repair naturally occurring DNA damage, in particular double-strand breaks (DSBs). Unrepaired DSBs can give rise to chromosomal instability which, in turn, can lead to loss of transgenes from the genome. As a consequence, protein titer can drop significantly, rendering protein production unprofitable. The invention provides a set of mutated DNA repair genes whose restoration yields significant improvement in DSB repair, genome stability, and protein titer.

### FIELD OF THE INVENTION

The present invention relates to methods to stabilize mammalian cells for recombinant protein production.

### BACKGROUND OF THE INVENTION

Chinese Hamster Ovary (CHO) cells have been the leading expression system for the industrial production of therapeutic proteins for over 30 years, and projections show they will maintain this dominant position into the foreseeable future, since they produce >80% of therapeutic proteins approved between 2014-18 [1]. Steady improvements in cell line development, media formulation, and bioprocessing now enable production yields exceeding 10 g/L, and sophisticated design strategies now produce high quality product with consistent post-translational modifications [2, 3]. Emerging tools and resources further enhance the success of CHO as the leading expression system, including the CHO and hamster genome sequencing efforts we led [4-6] and the implementation of genome editing tools [7-9]. These tools combined with genomics, systems biology, and other ‘omics resources now allow researchers to rely less on largely empirical, “trial-and-error” approaches to CHO cell line development, and move towards a more rational engineering approach, in pursuit of novel CHO lines with tailored, superior attributes [10-13].

Among cell attributes requiring further research and engineering, cell line instability, i.e. the propensity of a cell to lose valuable properties over time, remains a complex and frustrating problem since it can reverse earlier optimization efforts required to achieve other superior cell line attributes. One essential attribute, cell line instability, reverses is high productivity, leading to production instability, i.e. the significant decline in product titer following a few generations in culture. This major concern in industrial manufacturing quickly renders the production cycle unprofitable. Thus, typical cell line development pipelines must screen many clones prior to the actual production cycle to identify a “stable” producer (i.e., losing less than 30% of the initial titer during 60 generations [14]). These experiments are onerous and time-consuming, and even “stable” producers, due to the inevitable (yet slower) decline in productivity, are not economically viable over long culturing periods. Thus, cell line instability renders therapeutic protein production inefficient and contributes to high production costs and, consequently, high drug prices. Furthermore, the necessary assays take months to complete, thus, potentially prolonging the time to market, which delays the potential to treat patients and has major financial implications since it opens the door to loss of revenue from competing drugs and time for patent protected revenue, which could be billions of USD per month.

Most reported production instability cases are connected to two phenomena: (i) the loss of transgene copy numbers from the genome [15-23], or (ii) transcriptional transgene silencing through epigenetic mechanisms, such as promoter methylation or histone acetylation [18, 20, 24, 25]. Here, we address the problem of transgene loss, which commonly occurs and leads to non-producing subpopulations. Since massive transgene expression imposes a high metabolic demand on the host cell, such non-producing subpopulations will quickly outcompete producers in the cell pool, resulting in a net decline in titer.

It is widely understood that the loss of transgene copy number is likely caused by the instability of the CHO genome. Genomic instability involves the accelerated accumulation of mutations over short periods of time. This includes single-nucleotide polymorphisms (SNPs), short insertions & deletions (InDels), and chromosomal aberrations, such as translocations or loss of chromosomal segments. In CHO, chromosomal aberrations (also called “chromosomal instability”) was first reported in the 1970s when direct observations of CHO chromosomes revealed a divergence from the Chinese Hamster (Cricetulus griseus) karyotype and a variation in karyotype even among CHO clones [26]. Recent work has assayed the chromosomal aberrations in greater detail across several CHO lines [27], and demonstrated that the karyotype changes arise rapidly in culture [28]. These karyotype changes occur irrespective of growth condition, and do not differ markedly between pooled and clonal populations [29-31]. Loss of chromosomal material and improper chromosome fusions (translocations) are thought to be caused by one particularly critical mutation type, double-strand breaks (DSBs) [32, 33]. DSBs occur from ionizing radiation, attack by free radicals, or collapsed DNA replication forks [33]. Due to their potential fatal outcome on chromosomal integrity, eukaryotes are equipped with a complex set of molecular mechanisms to repair DSBs with little or no sequence loss [34, 35]. It follows that production instability due to transgene loss is likely from insufficient repair of DSBs in CHO.

While a mechanistic understanding of the underlying sources of production instability is emerging, it has been challenging to develop effective counter-strategies in mammalian cell bioprocessing. Detailed quantification of chromosomal instabilities in production cell lines has indicated that certain chromosome sites are less prone to instability than others [36]. This observation has suggested that transgene loss may be avoided by targeting transgenes to these stable chromosomal areas, an option now possible through the development of targeted transgene integration techniques [37-40]. Further studies used gene knock-outs (ATR and BRCA1, respectively) to increase product titer by increasing transgene copy number amplification [41, 42], but whether these knock-outs are able to sustain high production in long-term culture has remained questionable.

A pressing need remains for novel approaches to mitigate or counteract production instability stemming from double-strand breaks. In particular, we need strategies that are sufficiently generic to be easily applied across diverse CHO production lines. Although the mechanistic connections between production instability, chromosomal instability, and the occurrence of DNA damage (in particular DSBs) are becoming increasingly evident, the field has not systematically explored the engineering of DNA repair as a possible means to reduce transgene loss and production instability in CHO. The above-mentioned report of ATR as a target to improve production stability is interesting in this context because this gene is a well-known component of the cellular DSB response [43]. Inactivation of this gene resulted in an increase in transgene copies during the amplification phase, but also a less rigid cell cycle control and higher chromosomal instability, which may exacerbate production instability in the long run [41]. Therefore, rather than inactivating DNA repair genes for short-term gains, enhancement of DNA repair could constitute a promising approach to achieve long-term improvement in production stability.

### OBJECT OF THE INVENTION

It is an object of embodiments of the invention to provide methods and cells for better and more stable production of recombinant proteins.

### SUMMARY OF THE INVENTION

It has been found by the present inventor(s) that by reversing mutations or reversing the silencing of certain genes involved in DNA repair mechanisms of the cell, such a cell may be a better and more stable producer of recombinant proteins produced in such a modified cell.

So, in a first aspect the present invention relates to a method of preparing a cell for expression of a gene of interest, comprising reverting a mutation or a silencing of one or more DNA repair gene in the cell. One specific aspect relates to a method of preparing a cell for expression of a gene of interest, comprising reverting a mutation in a DNA repair gene in the cell. Another specific aspect relates to a method of preparing a cell for expression of a gene of interest, comprising the reversing of a silencing of one or more DNA repair gene in the cell.

In a second aspect the present invention relates to a cell made by the methods of the invention.

In a further aspect the present invention relates to a method of producing a gene product comprising expressing a gene of interest in a cell made by the method of the invention, and purifying the gene product.

In a further aspect the present invention relates to a double-stranded break (DSB) reporter system providing quantitative detection of DSB repair efficiency in living cells In embodiments, the invention provides methods and compositions for increased expression or restoration of DNA repair genes in a host cell for recombinant protein production.

In other embodiments the methods of preparing a cell for expression of a gene of interest, comprising reverting a mutation in a DNA repair gene in the cell.

The invention provides methods of preparing a cell for expression of a gene of interest, wherein the gene of interest has an increased expression level, compared to the expression in the unmodified cell.

The invention provides methods of preparing a cell for expression of a gene of interest, wherein the cell has improved double strand break repair and/or genome stability, compared to the expression in the unmodified cell.

The invention provides methods of preparing a cell for expression of a gene of interest, wherein the cell has improved protein product titer, compared to the expression in the unmodified cell.

The invention provides methods of preparing a cell for expression of a gene of interest, wherein the genes targeted are among the DNA repair machinery provided herein.

The invention provides methods of preparing a cell for expression of a gene of interest, wherein the DNA repair gene is ATM (R2830H) and/or PRKDC (D1641N).

The invention provides methods of preparing a cell for expression of a gene of interest, wherein the DNA repair gene is MCM7, PPP2R5A, P1A54, PBRM1, and/or PARP2. The invention provides methods of preparing a cell for expression of a gene of interest, wherein the mutation includes SNPs and/or indels in CHO cells, as provided herein.

The invention provides methods of preparing a cell for expression of a gene of interest, wherein the gene has decreased expression in CHO cells, compared to native hamster tissue.

The invention provides a method of producing a gene product comprising expressing a gene of interest in a cell made by the methods described herein, and purifying the gene product.

The invention also provides a double-stranded break (DSB) reporter system providing quantitative detection of DSB repair efficiency in living cells as described herein.

### LEGENDS TO THE FIGURE

FIGS. 1A-1D show identification of SNPs in DNA repair genes. FIG. 1A shows an analysis of whole-genome sequencing data from 11 major CHO cell lines identified a total of 157 SNPs across a broad range of DNA repair categories (Gene Ontology classes). The number of CHO lines affected (x-axis) and SNP deleteriousness (y-axis: Negative PROVEAN score) are averaged across all mutations detected in each category. Dashed line indicates the recommended threshold (2.282) to separate neutral from detrimental SNPs [54]. FIG. 1B shows SNPs that have undergone loss of heterozygosity (LOH) (i.e., absence of the Chinese hamster wildtype allele at that locus). FIG. 1C shows SNPs further evaluated and having undergone LOH in genes for which (at least partial) relevance to double-strand break (DSB) repair has been described. FIG. 1D shows data from FIG. 1C with individual SNPs are shown.

FIGS. 2A-2B show GFP-based double-strand break (DSB) reporter system. FIG. 1A shows Step 1: The GFP expression cassette, comprising a promoter, a large (2 kb) spacer, and a GFP reading frame, is integrated into the genome of the cell line to be analyzed. The spacer prevents the promoter from driving GFP expression. Step 2: Transient transfection with the DSB-inducing plasmid (B) induces two DSBs at the 5′ and 3′ ends of the spacer. Successfully transfected cells are identified through far-red fluorescence from miRFP670, fused to Cas9 (B). Step 3: Transfected cells that repair both DSBs properly keep the spacer in place and thus remain GFP-negative. Transfected cells that fail to repair both DSBs in time produce a large sequence loss, moving the GFP in proximity to the promoter, resulting in GFP expression. Thus, the fraction of GFP-positive cells among all transfected cells (far-red positive) serves as a read-out for the inefficiency of DSB repair. Assay modified from [55]. FIG. 2B shows the DSB-triggering plasmid used comprises two sgRNAs targeting both ends of the 2 kb spacer, and a Cas9 reading frame, fused to the far-red fluorescent protein miRFP670.

FIG. 3 shows validation of the GFP reporter system for quantification of DSB repair. Flow cytometry analysis of 10,000 CHO-K1 cells carrying the GFP reporter system after either mock transfection (upper left), DSB-inducer transfection (lower left), and DBS-inducer transfection with simultaneous inhibition of the ATM kinase (lower right) (3 μM KU-20019, Sellenckchem). ATM inhibition increases the fraction of GFP+ cells (upper right), confirming the validity of the assay. FACS analysis carried out 24h after transfection. SSC-H: Side-scatter. n=2; t-test.

FIGS. 4A-4B show restoration of DNA repair genes improves DSB repair in CHO. FIG. 4A shows flow cytometry analysis of 50,000 cells of CHO-K1, CHO-K1 ATM+/+(reverted R2830H), and CHO-K1 ATM+/+ PRKDC+/+ (reverted R2830H and reverted D1641N), expressing the GFP reporter system (FIG. 2) after transfection with the DSB-inducer plasmid. FACS carried out 24h after transfection. FIG. 4B shows the same analysis with 50,000 cells of CHO-SEAP wt, and CHO-SEAP overexpressing Chinese Hamster xrcc6.

FIG. 5: SNP reversal and DSB reporter assay. (a): Left: SNP reversal is carried out by targeting an sgRNA to a PAM (NGG, reverse strand displayed) proximal to the respective SNP (red). A ssDNA homology donor oligo carrying the reversed base (red) is provided as a repair template. The donor oligo carries additional, silent SNPs (green) to prevent re-targeting of the repaired sequence. Right: Sequence alignment of targeted SNP loci in ATM (R2830H, top) and PRKDC (D1641N, bottom). CHO-K1: host strain, Donor: homology oligo template, ATM+/PRKDC+: cell clones obtained from SNP reversal (PRKDC+ is short for ATM+ PRKDC+ as PRKDC D1641N was restored in the ATM+ cell line), C. gri: Chinese Hamster (Cricetulus griseus). (b): Step 1: The EJ5-GFP cassette comprises a promoter, a 2 kb spacer, and a GFP reading frame. The spacer prevents the promoter from driving GFP expression. The cassette is integrated into the host genome. Step 2: Transient transfection with a DSB-inducing plasmid, encoding Cas9 and two sgRNAs, targets two sites at the 5′ and 3′ ends of the spacer. Successfully transfected cells are identified through far-red fluorescence of the Cas9:miRFP670 fusion. Step 3: Transfected cells that repair both DSBs properly keep the spacer in place and remain GFP-negative. Loss of the spacer due to compromised DNA repair moves the GFP in proximity to the promoter, resulting in positive GFP expression (assay modified from [84]). (c): Top: DSB repair ability is quantified through flow cytometry by relating the fraction of GFP-positive cells to all transfected cells, with the gates shown. Bottom: Flow cytometry analysis of CHO-K1 wildtype cells carrying EJ5-GFP after transfection with the DSB-inducing plasmid (b). Cells were supplemented with DMSO (middle) or treated with a chemical inhibitor against the ATM kinase (right) (KU-20019 3 μM). Data showing pooled populations from three independent transfections per condition. Untransfected wildtype cells were used as control (left). Green dashed line: GFP intensity threshold. Two-sample Kolmogorov-Smirnov tests (*** p<0.001; n>6,900 cells) FIG. 6: Quantification of DSB repair ability in engineered CHO cells. (a): EJ5-GFP assay on CHO-K1 wildtype, ATM+ and ATM+ PRKDC+ cell lines. Data showing pooled populations from two independent transfections per cell line. Untransfected wildtype cells were used as control (left). Green dashed line: GFP intensity threshold. Two-sample Kolmogorov-Smirnov tests (*** p<0.001; n>6,700 cells). (b): Immunostainings against γH2AX in CHO-K1 wildtype, ATM+, ATM+ PRKDC+. y-axis shows accumulated γH2AX signal, normalized by nuclear size (log-transformed). t-tests (*** p<0.001; n>114 nuclei). Whiskers showing 5/95-quantiles. Cells counterstained with DAPI.

FIG. 7: Quantification of genome fragmentation in engineered CHO cells. (a): Representative composite images of wildtype, ATM+ and ATM+ PRKDC+ cells after electrophoresis in a low-melting agar (comet assay). Nuclei stained with Vista DNA Green (Abcam). (b): Quantification of comet assay data using both tail length and tail moment (=tail length*DNA in tail [%]) of untreated cells (left), cells treated with X-ray radiation (middle), and cell treated with bleomycin (right). t-tests (ns: not significant; ** p<0.01; ** *** p<0.001; n>53 nuclei). Whiskers showing 5/95-quantiles.

FIG. 8: Karyotype analysis after long-term culture. (a): Main karyotype after 60 passages. Chromosomes were identified using pseudo-color probes, specific for each Cricetulus griseus chromosome. (b): Examples for deviating karyotypes in WT (top) and WT, supplemented with the ATM inhibitor KU-60019 (bottom). Open arrows indicate a numerical variation (i.e. gain/loss of a chromosome), closed arrows indicate a structural variation (i.e. an altered color pattern). (c): Left: Classification of karyotypes into: showing at least one numerical variation with no structural variations (grey), showing at least one structural variation with no numerical variations (red), showing both at least one numerical and at least one structural variation (grey/red striped), and showing no variations (white), relative to the main karyotype (a). Differences in frequency of structural variations (red and red/grey fractions) significant at 5% level (Binomial test) (asterisks omitted for clarity). Averaged fractions from duplicate experiments: WT n=26/34; ATM+n=21/37; ATM+ PRKDC+n=21/37; WT+KU60019 n=8/19. Right: Total number of chromosomes per karyotype. Bar=median. Non-parametric ANOVA (Kruskal-Wallis test).

FIG. 9: DSB repair and protein titer stability in a producing CHO cell line. (a): EJ5-GFP assay on CHO-SEAP wildtype, CMV::XRCC6, CMV::XRCC6 ATM+ PRKDC+ cell lines, and CMV::XRCC6 cells, supplemented with the ATM inhibitor KU-60019. Data showing pooled populations from two independent transfections per cell line. Untransfected wildtype cells were used as control (right). Green dashed line: GFP intensity threshold. Two-sample Kolmogorov-Smirnov tests (*** p<0.001; n>3,800 cells). (b): The transgene expression cassette comprises both secreted alkaline phosphatase (SEAP) and dihydrofolate reductase (DHFR), an essential metabolic enzyme. Methotrexate (MTX) is a competitive inhibitor of DHFR and is used as a selector against loss of the cassette in culture. (c): Sketch of the long-term culture experiment. Both CHO-SEAP wildtype and CMV::XRCC6 cell lines were supplemented with 5 μM MTX for 2 weeks to select for high SEAP expression after which only one sample per cell line was maintained under MTX supplementation for another 14 weeks. Samples were cultured in duplicates. (d): Left: Total SEAP titer (PhosphaLight assay, Thermo Fischer) in indicated cell lines at different passages. Right: SEAP titer normalized to cell count in indicated cell lines at different passages (n>4). Blank sample indicates media only.

### DETAILED DISCLOSURE OF THE INVENTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2nd ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20th ed., (Lippincott, Williams & Wilkins 2003), and Remington, The Science and Practice of Pharmacy, 22th ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

It is understood that aspects and embodiments of the invention described herein include “consisting” and/or “consisting essentially of” aspects and embodiments.

As used herein, the transitional phrases “consists essentially of” and “consisting essentially of” are used to define a protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

“Amplification” refers to any known procedure for obtaining multiple copies of a target nucleic acid or its complement, or fragments thereof. The multiple copies may be referred to as amplicons or amplification products. Amplification, in the context of fragments, refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), reverse transcription polymerase chain reaction (RT-PCR), ligase chain reaction (LCR), strand-displacement amplification (SDA), and transcription-mediated or transcription-associated amplification. Amplification is not limited to the strict duplication of the starting molecule. For example, the generation of multiple cDNA molecules from RNA in a sample using reverse transcription (RT)-PCR is a form of amplification.

Furthermore, the generation of multiple RNA molecules from a single DNA molecule during the process of transcription is also a form of amplification. During amplification, the amplified products can be labeled using, for example, labeled primers or by incorporating labeled nucleotides.

“Amplicon” or “amplification product” refers to the nucleic acid molecule generated during an amplification procedure that is complementary or homologous to a target nucleic acid or a region thereof. Amplicons can be double stranded or single stranded and can include DNA, RNA or both. Methods for generating amplicons are known to those skilled in the art.

“Codon” refers to a sequence of three nucleotides that together form a unit of genetic code in a nucleic acid.

“Codon of interest” refers to a specific codon in a target nucleic acid that has diagnostic or therapeutic significance (e.g. an allele associated with viral genotype/subtype or drug resistance).

“Complementary” or “complement thereof” means that a contiguous nucleic acid base sequence is capable of hybridizing to another base sequence by standard base pairing (hydrogen bonding) between a series of complementary bases. Complementary sequences may be completely complementary (i.e. no mismatches in the nucleic acid duplex) at each position in an oligomer sequence relative to its target sequence by using standard base pairing (e.g., G:C, A:T or A:U pairing) or sequences may contain one or more positions that are not complementary by base pairing (e.g., there exists at least one mismatch or unmatched base in the nucleic acid duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Downstream” means further along a nucleic acid sequence in the direction of sequence transcription or read out.

“Upstream” means further along a nucleic acid sequence in the direction opposite to the direction of sequence transcription or read out.

“Polymerase chain reaction” (PCR) generally refers to a process that uses multiple cycles of nucleic acid denaturation, annealing of primer pairs to opposite strands (forward and reverse), and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. There are many permutations of PCR known to those of ordinary skill in the art.

“Position” refers to a particular amino acid or amino acids in a nucleic acid sequence.

“Primer” refers to an enzymatically extendable oligonucleotide, generally with a defined sequence that is designed to hybridize in an antiparallel manner with a complementary, primer-specific portion of a target nucleic acid. A primer can initiate the polymerization of nucleotides in a template-dependent manner to yield a nucleic acid that is complementary to the target nucleic acid when placed under suitable nucleic acid synthesis conditions (e.g. a primer annealed to a target can be extended in the presence of nucleotides and a DNA/RNA polymerase at a suitable temperature and pH). Suitable reaction conditions and reagents are known to those of ordinary skill in the art. A primer is typically single stranded for maximum efficiency in amplification, but may alternatively be double stranded. If double stranded, the primer is generally first treated to separate its strands before being used to prepare extension products. The primer generally is sufficiently long to prime the synthesis of extension products in the presence of the inducing agent (e.g. polymerase). Specific length and sequence will be dependent on the complexity of the required DNA or RNA targets, as well as on the conditions of primer use such as temperature and ionic strength. Preferably, the primer is about 5-100 nucleotides. Thus, a primer can be, e.g., 5, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95 or 100 nucleotides in length. A primer does not need to have 100% complementarity with its template for primer elongation to occur; primers with less than 100% complementarity can be sufficient for hybridization and polymerase elongation to occur. A primer can be labeled if desired. The label used on a primer can be any suitable label, and can be detected by, for example, spectroscopic, photochemical, biochemical, immunochemical, chemical, or other detection means. A labeled primer therefore refers to an oligomer that hybridizes specifically to a target sequence in a nucleic acid, or in an amplified nucleic acid, under conditions that promote hybridization to allow selective detection of the target sequence.

A primer nucleic acid can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

“Region” refers to a portion of a nucleic acid wherein said portion is smaller than the entire nucleic acid.

“Region of interest” refers to a specific sequence of a target nucleic acid that includes all codon positions having at least one single nucleotide substitution mutation associated with a genotype and/or subtype that are to be amplified and detected, and all marker positions that are to be amplified and detected, if any.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) ““Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

“Fragment” refers to a piece of contiguous nucleic acid that contains fewer nucleotides than the complete nucleic acid.

“Hybridization,” “annealing,” “selectively bind,” or “selective binding” refers to the base-pairing interaction of one nucleic acid with another nucleic acid (typically an antiparallel nucleic acid) that results in formation of a duplex or other higher-ordered structure (i.e. a hybridization complex). The primary interaction between the antiparallel nucleic acid molecules is typically base specific, e.g., A/T and G/C. It is not a requirement that two nucleic acids have 100% complementarity over their full length to achieve hybridization. Nucleic acids hybridize due to a variety of well characterized physio-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

“Nucleic acid” or “nucleic acid molecule” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid backbone can be made up of a variety of linkages, including one or more of sugar-phosphodiester linkages, peptide-nucleic acid bonds, phosphorothioate linkages, methylphosphonate linkages, or combinations thereof. Sugar moieties of the nucleic acid can be ribose, deoxyribose, or similar compounds having known substitutions (e.g. 2′-methoxy substitutions and 2′-halide substitutions). Nitrogenous bases can be conventional bases (A, G, C, T, U) or analogs thereof (e.g., inosine, 5-methylisocytosine, isoguanine). A nucleic acid can comprise only conventional sugars, bases, and linkages as found in RNA and DNA, or can include conventional components and substitutions (e.g., conventional bases linked by a 2′-methoxy backbone, or a nucleic acid including a mixture of conventional bases and one or more base analogs). Nucleic acids can include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA). Nucleic acids can include modified bases to alter the function or behavior of the nucleic acid (e.g., addition of a 3′-terminal dideoxynucleotide to block additional nucleotides from being added to the nucleic acid). Synthetic methods for making nucleic acids in vitro are well known in the art although nucleic acids can be purified from natural sources using routine techniques. Nucleic acids can be single-stranded or double-stranded.

A nucleic acid is typically single-stranded or double-stranded and will generally contain phosphodiester bonds, although in some cases, as outlined, herein, nucleic acid analogs are included that may have alternate backbones, including, for example and without limitation, phosphoramide (Beaucage et al. (1993) Tetrahedron 49(10):1925 and references therein; Letsinger (1970) J. Org. Chem. 35:3800; Sprinzl et al. (1977) Eur. J. Biochem. 81:579; Letsinger et al. (1986) Nucl. Acids Res. 14: 3487; Sawai et al. (1984) Chem. Lett. 805; Letsinger et al. (1988) J. Am. Chem. Soc. 110:4470; and Pauwels et al. (1986) Chemica Scripta 26: 1419, which are each incorporated by reference), phosphorothioate (Mag et al. (1991) Nucleic Acids Res. 19:1437; and U.S. Pat. No. 5,644,048, which are both incorporated by reference), phosphorodithioate (Briu et al. (1989) J. Am. Chem. Soc. 111:2321, which is incorporated by reference), O-methylphosphoroamidite linkages (see Eckstein, Oligonucleotides and Analogues: A Practical Approach, Oxford University Press (1992), which is incorporated by reference), and peptide nucleic acid backbones and linkages (see, Egholm (1992) J. Am. Chem. Soc. 114:1895; Meier et al. (1992) Chem. Int. Ed. Engl. 31:1008; Nielsen (1993) Nature 365:566; and Carlsson et al. (1996) Nature 380:207, which are each incorporated by reference). Other analog nucleic acids include those with positively charged backbones (Denpcy et al. (1995) Proc. Natl. Acad. Sci. USA 92:6097, which is incorporated by reference); non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook; Mesmaeker et al. (1994) Bioorganic & Medicinal Chem: Lett. 4: 395; Jeffs et al. (1994) J. Biomolecular NMR 34:17; and Tetrahedron Lett. 37:743 (1996), which are each incorporated by reference) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook, which references are each incorporated by reference. Nucleic acids containing one or more carbocyclic sugars are also included within the definition of nucleic acids (see Jenkins et al. (1995) Chem. Soc. Rev. pp 169-176, which is incorporated by reference). Several nucleic acid analogs are also described in, e.g., Rawls, C & E News Jun. 2, 1997 page 35, which is incorporated by reference. These modifications of the ribose-phosphate backbone may be done to facilitate the addition of additional moieties such as labels, or to alter the stability and half-life of such molecules in physiological environments.

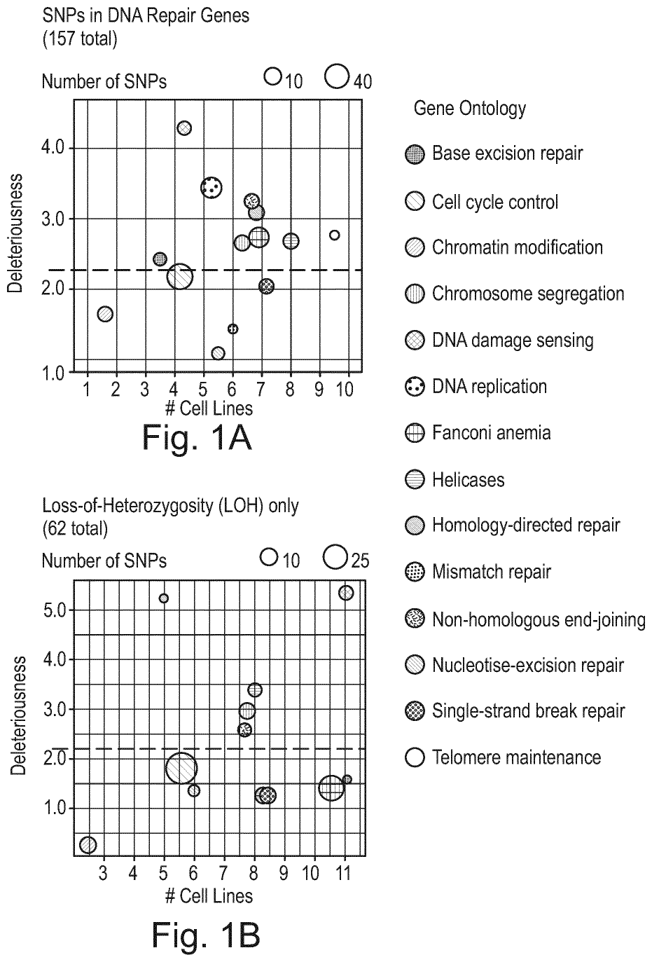

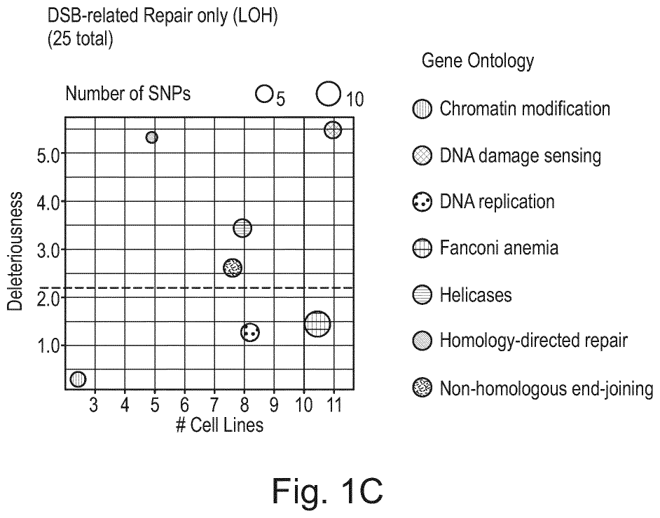

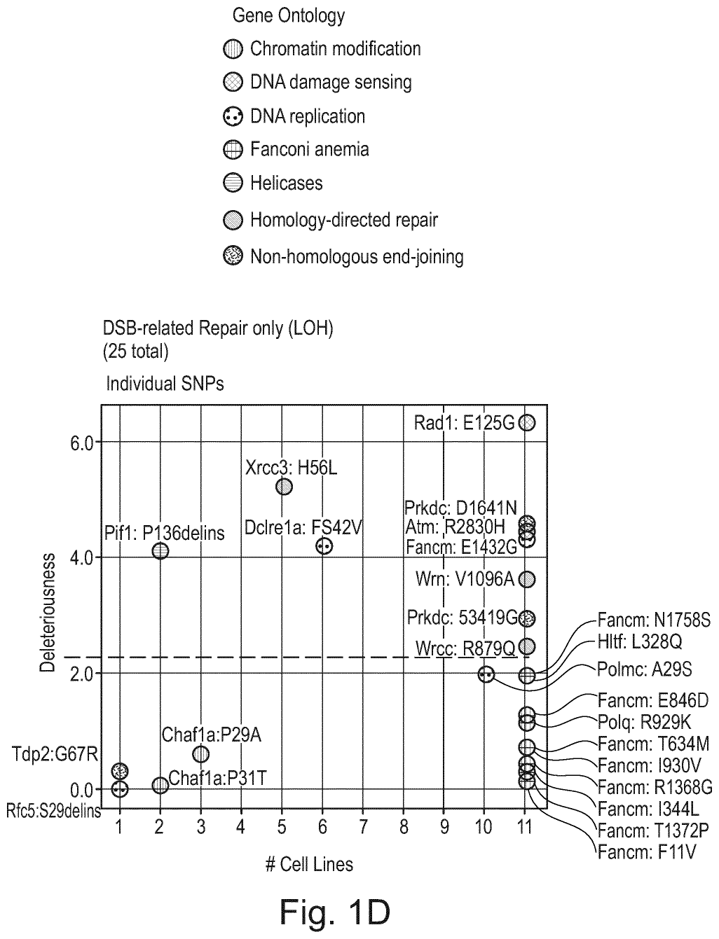

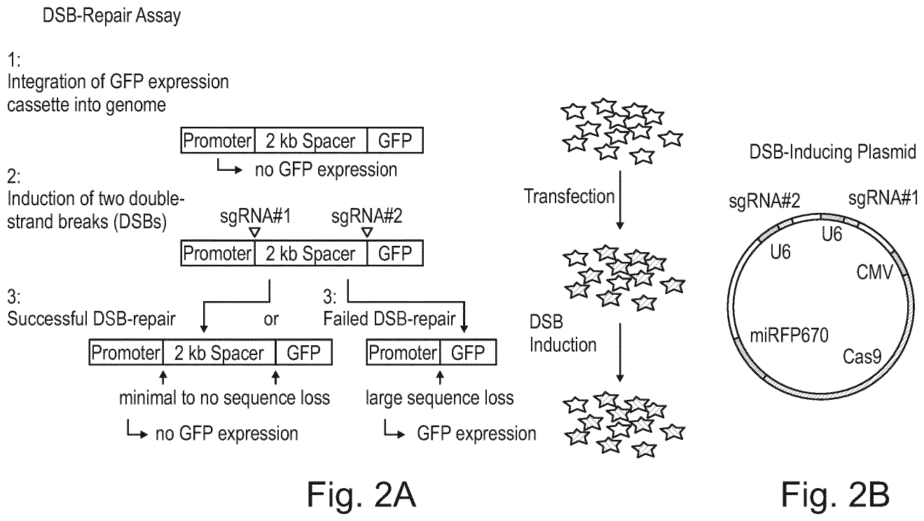

The disclosure provides a detection of mutations in DNA repair genes. We have analyzed whole-genome sequencing data from 11 CHO cell lines, including those commonly used for cell line development in biopharmaceutical production (e.g. CHO-S, CHO-XB11, CHO-DG44) and aligned them to the recent Chinese Hamster genome assembly [5]. Sequencing analysis of DNA repair genes has revealed a total of 157 SNPs in DNA repair genes across 11 major CHO cell lines. These genes span 14 ontology categories related to DNA repair (FIG. 1A). Among these, 62 SNPs show a loss of heterozygosity (FIG. 1B). The predicted deleteriousness of these SNPs varied between −0.005 and −8.821 (PROVEAN scores), with a total of 19 SNPs being predicted as detrimental (FIG. 1B, dashed line). In particular, we found several detrimental SNPs in genes associated with DSB repair (FIG. 2C, D).

The invention provides a tool to quantify double-strand break (DSB) repair in CHO. We have implemented a DSB reporter system (based on the EJ5-GFP tool provided in [44]) in both CHO-K1 and CHO-SEAP, an alkaline phosphatase producing cell line [45]. This reporter system comprises a GFP reading frame, separated from its promoter with a large (2 kb) spacer (FIG. 2A). Expression of two sgRNAs creates DSBs at the 5′ and 3′ end of the spacer (FIG. 2A,B); in the case of inefficient DSB repair, the spacer will often be lost in a large deletion, thus putting the GFP in proximity to its promoter, resulting in positive GFP expression. Successful DSB repair will keep the spacer in place and the GFP expression will stay negative (FIG. 2A). Thus, this tool allows quantitative detection of DSB repair efficiency in living cells and is a powerful read-out for how restoration of individual DSB repair genes improves chromosome stability.

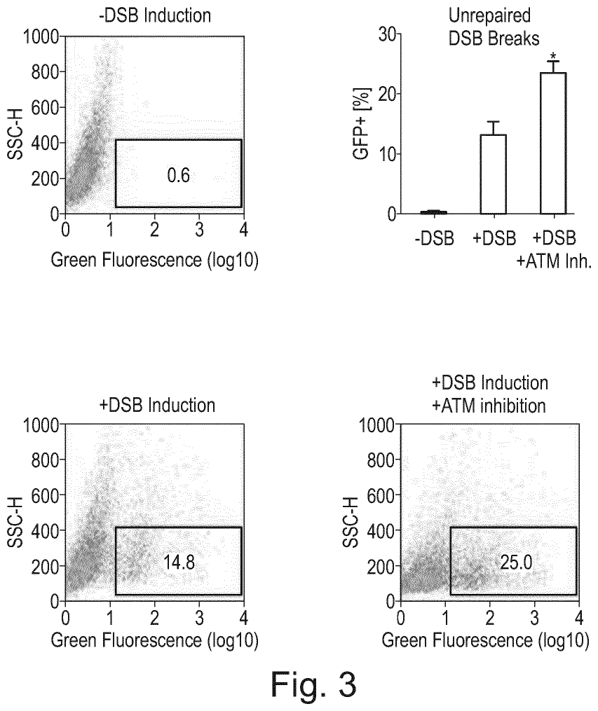

We have successfully generated clonal populations carrying the DSB reporter system that quantifies the efficacy of double strand break repair (FIG. 2A). 24h after transfection with the DSB-inducer (FIG. 2B), significant increases in GFP+ signal can be detected, corroborating the notion of insufficient DSB repair in CHO cells (FIG. 3). Furthermore, we treated cells with a chemical inhibitor against the ATM kinase, which is considered one of the most upstream cellular responses to DSBs [46]. We saw a significant increase in the fraction of GFP+ cells when running the GFP expression assay (FIG. 3), consistent with the central role of ATM in DSB repair.

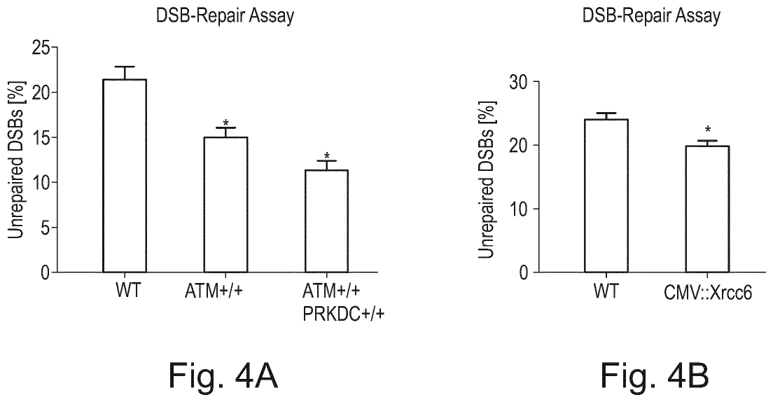

Restoration of DNA repair genes. We successfully reverted two SNPs, ATM R2830H and PRKDC D1641N, both predicted to be highly detrimental by our variant analysis (FIG. 1D). Both reversals were done in succession in the same cell line to assess the cumulative effect of DNA repair improvements. We saw noticeable improvement in DSB repair capability after reversal of ATM R2830H (ATM+/+: FIG. 4A), which confirms the classification of ATM R2830H as a detrimental SNP. Moreover, the observation that DSB repair deficiency was still significantly exacerbated upon ATM inhibition (FIG. 4A) in wildtype CHO-K1 indicates that the nature of the R2830H allele is hypomorphic, rather than a full loss-of-function—a conclusion that likely will apply to most SNPs found in our analysis. Reversal of PRKDC D1641N further improved DSB repair (ATM+/+ PRKDC+/+; FIG. 4A), in accordance with the notion that gradual restoration of DNA repair capability can be achieved by successive restoration of DNA repair genes. In addition, we introduced a Chinese Hamster sequence of the DNA repair gene xrcc6 which also lead to a noticeable increase in DNA repair capability (FIG. 4B).

### Specific Embodiments of the Invention

The present invention relates to a method of preparing a cell for expression of a gene of interest, comprising reverting a mutation or a silencing of one or more DNA repair gene in the cell.

In some embodiments the gene of interest has an increased expression level, compared to the expression in the unmodified cell.

In some embodiments the cell has improved double strand break repair and/or genome stability, compared to the expression in the unmodified cell.

In some embodiments the the cell has improved protein product titer, compared to the expression in the unmodified cell.

In some embodiments the the one or more DNA repair gene targeted by reverting mutation are among the DNA repair machinery provided herein, such as any one or more of table 3.

In some embodiments the the one or more DNA repair gene is selected from any one of XRCC6, ATM and/or PRKDC, such as any one of mutation XRCC6 (Q606H), ATM (R2830H) and/or PRKDC (D1641N).

In some embodiments the one or more DNA repair gene is targeted for reversing a silencing, such as any one DNA repair gene selected from MCM7, PPP2R5A, PIAS4, PBRM1, and/or PARP2.

In some embodiments the mutation includes SNPs and/or indels in CHO cells, as provided herein.

In some embodiments the one or more DNA repair gene has decreased expression in CHO cells, compared to native hamster tissue.

In some embodiments the one or more DNA repair gene is one, at least two, at least three, at least four, at least five, at least six, at least 7, at least 8, at least 9, or at least 10 DNA repair genes.

In some embodiments the cell is a CHO cell, such as a CHO cell selected from any one of table 1, such as CHO-K1, CHO-K1/SF, CHO protein-free, CHO-DG44, CHO-S, C0101, CHO—Z, CHO-DXB11, and CHO-pgsA-745.

### Example 1

### Methods

### Detection of Mutations in DNA Repair Genes

To test the mutational burden in DNA repair genes in a broad panel of cell lines used in biopharmaceutical production, whole-genome sequencing data of 11 CHO cell lines (Table 1) were analyzed and compared to the Chinese Hamster genome [5, 6]. Raw sequencing reads were pre-processed using fastQC [47] for quality control and Trimmomatic [48] to remove low-quality base pairs and adapters. The reads were aligned to the Chinese Hamster genome using BWA [49]. Non-synonymous SNPs and InDels were called using the gatk3.5 software package [50] using standard parameters and annotated using SnpEff [51]. SnpSift [52] was used to filter genes with ontologies related to DNA repair [53]. The PROVEAN tool [54] served to predict deleteriousness of each mutation. Finally, gene targets were prioritized based on a metric combining the PROVEAN score, the heterozygosity, the number of CHO cell lines affected by this SNP, and their relevance for certain DNA-repair pathways (as reported in the literature).

TABLE 1
CHO cell lines analyzed

| | | NCBI Sequence |
|---|---|---|
| | | Read Archive |
| Cell line | Origin | Number |
| CHO-K1 | ATCC | SRP045758 |
| CHO-K1 | ECACC | SRS406579 |
| CHO-K1/SF | ECACC | SRS406580 |
| CHO protein- | ECACC | SRS406578 |
| free | | |
| CHO-DG44 | Life Technologies | SRS406582 |
| CHO-S | Life Technologies | SRS406581 |
| CHO-S | Clone from the Technical | (Unpublished) |
| | University of Denmark (derived | |
| | from Life Technologies) | |
| C0101 | Undisclosed company (Drug | SRX258098 |
| | producing cell line derived from | |
| | CHO-S from Life Technologies) | |
| CHO-Z | Clone from the Technical | (Unpublished) |
| | University of Denmark (Serum- | |
| | free suspension adapted clone | |
| | derived from an ECACC CHO-K1 | |
| | clone) | |
| CHO-DXB11 | Clone from the Technical | SRX689758 |
| | University of Denmark | |
| CHO-pgsA- | ATCC | (Unpublished) |
| 745 | | |

To detect genes that have been silenced in CHO cells, one must quantify gene transcription in the native Chinese hamster tissues and compare the expression to CHO cells. For this we quantified gene transcription in multiple tissues from the hamster using several technologies that measure transcriptional levels at the start of the mRNA (transcription start sites (TSSs) and mRNA levels throughout the genes. These are described as follow

Quantifying Transcription Start Sites (TSSs) of genes: Sequencing data used here is Transcription Start Site sequencing, which measures RNA at the start of the transcripts. The methods include capped small RNA sequencing (csRNA-seq) and 5′ Global Nuclear Run On Sequencing (5′GRO-seq).

Sample preparation: Female Chinese hamsters (Cricetulus griseus) were generously provided by George Yerganian (Cytogen Research and Development, Inc) and housed at the University of California San Diego animal facility on a 12h/12h light/dark cycle with free access to normal chow food and water. All animal procedures were approved by the University of California San Diego Institutional Animal Care and Use Committee in accordance with University of California San Diego research guidelines for the care and use of laboratory animals. None of the used Hamsters were subject to any previous procedures and all of them were used naively, without any previous exposure to drugs. Euthanized hamsters were quickly chilled in a wet ice/ethanol mixture (˜50/50), organs were isolated, placed into Trizol LS, flash frozen in liquid nitrogen and stored at −80 C for later use. CHO-K1 cells were grown in F-K12 medium (GIBCO-Invitrogen, carlsbad, CA, USA) at 37° C. with 5% CO₂.

Bone marrow-derived macrophaqe (BMDM) culture: Hamster bone marrow-derived macrophages (BMDMs) were generated as detailed previously (99. Link et al. 2018). Femur, tibia and iliac bones were flushed with DMEM high glucose (Corning), red blood cells were lysed, and cells cultured in DMEM high glucose (50%), 30% L929-cell conditioned laboratory-made media (as source of macrophage colony-stimulating factor (M-CSF)), 20% FBS (Omega Biosciences), 100 U/ml penicillin/streptomycin+L-glutamine (Gibco) and 2.5 μg/ml Amphotericin B (HyClone). After 4 days of differentiation, 16.7 ng/ml mouse M-CSF (Shenandoah Biotechnology) was added. After an additional 2 days of culture, non-adherent cells were washed off with room temperature DMEM to obtain a homogeneous population of adherent macrophages which were seeded for experimentation in culture-treated petri dishes overnight in DMEM containing 10% FBS, 100 U/ml penicillin/streptomycin+L-glutamine, 2.5 μg/ml Amphotericin B and 16.7 ng/ml M-CSF. For Kdo2-Lipid A (KLA), activation, macrophages were treated with 10 ng/mL KLA (Avanti Polar Lipids) for 1 hour.

RNA-seq: RNA was extracted from organs that were homogenized in Trizol LS using an Omni Tissue homogenizer. After incubation at RT for 5 minutes, samples were spun at 21.000 g for 3 minutes, supernatant transferred to a new tube and RNA extracted following manufacturer's instructions. Strand-specific total RNA-seq libraries from ribosomal RNA-depleted RNA were prepared using the TruSeq Stranded Total RNA Library kit (Illumina) according to the manufacturer-supplied protocol. Libraries were sequenced 100 bp paired-end to a depth of 29.1-48.4 million reads on an Illumina HiSeq2500 instrument.

csRNA-seq Protocol: Capped small RNA-sequencing was performed identically as described by (95. Duttke et al. 2019). Briefly, total RNA was size selected on 15% acrylamide, 7M UREA and 1×TBE gel (Invitrogen EC6885BOX), eluted and precipitated over night at −80° C. Given that the RIN of the tissue RNA was often as low as 2, essential input libraries were generated to facilitate accurate peak calling. csRNA libraries were twice cap selected prior to decapping, adapter ligation and sequencing. Input libraries were decapped prior to adapter ligation and sequencing to represent the whole repertoire of small RNAs with 3′-OH. Samples were quantified by Qbit (Invitrogen) and sequenced using the Illumina NextSeq 500 platform using 75 cycles single end.

Global Run-On Nuclear Sequencing Protocol: Nuclei from hamster tissues were isolated as described in (98. Hetzel et al. 2016). Hamster BMDM nuclei were isolated using hypotonic lysis [10 mM Tris-HCl pH 7.5, 2 mM MgCl₂, 3 mM CaCl₂; 0.1% IGEPAL] and flash frozen in GRO-freezing buffer [50 mM Tris-HCl pH 7.8, 5 mM MgCl₂, 40% Glycerol]. 0.5-1×10⁶ BMDM nuclei were run-on with BrUTP-labelled NTPs as described (96. Duttke et al. 2015) with 3×NRO buffer [15 mM Tris-CI pH 8.0, 7.5 mM MgCl₂, 1.5 mM DTT, 450 mM KCl, 0.3 U/μl of SUPERase In, 1.5% Sarkosyl, 366 μM ATP, GTP (Roche) and Br-UTP (Sigma Aldrich) and 1.2 μM CTP (Roche, to limit run-on length to ˜40 nt)]. Reactions were stopped after five minutes by addition of 750 μl Trizol LS reagent (Invitrogen), vortexed for 5 minutes and RNA extracted and precipitated as described by the manufacturer.

GRO-seq: RNA was fragmented, and BrU enrichment was performed using a BrdU Antibody (Sigma B8434-200 μl Mouse monoclonal BU-33) coupled to Protein G (Dynal 1004D) beads. Beads were subsequently collected on a magnet. End-repair was done and a second round of BrU enrichment was done. Input libraries were decapped prior to adapter ligation and sequencing to represent the whole repertoire of small RNAs with 3′-OH. Samples were quantified by Qbit (Invitrogen) and sequenced using the Illumina NextSeq 500 platform using 75 cycles single end.

5′GRO-seq: RNA was dephosphorylated using 10 μl of dephosporylation MM [2 μl 10×CutSmart, 6.75 μl dH2O+T, 1 μl Calf Intestinal alkaline Phosphatase (10 U; CIP, NEB) or quick CIP (10 U, NEB), 0.25 μl SUPERase-In (5U)] was added. BrdU enrichment was performed as described for GRO-seq. A second round of dephosphorylation and BrdU enrichment were performed. Libraries were prepared as described in Hetzel et al. (2016). Briefly, libraries were done as described for GRO-seq (above) with exception of the 3′Adapter ligation step. Here, prior to 3′Adapter ligation, samples were dissolved in 3.75 μl TET heated to 70° C. for 2 minutes and placed on ice. RNAs were decapped by addition of 6.25 μl RppH MM [1 μl 10×T4 RNA ligase buffer, 4 μl 50% PEG8000, 0.25 μl SUPERase-In, 1 μl RppH (5U)] and incubated at 37° C. for 1 hour. 5′ adapter ligation, reverse transcription and library size selection were performed as described for GRO-seq. Samples were amplified for 14 cycles, size selected for 160-250 bp and sequenced on an Illumina NextSeq 500 at using 75 cycles single end.

RNA processing: Sequence data for all RNA-seq data was quality controlled using FastQC (v0.11.6. Babraham Institute, 2010), and cutadapt v1.16 (100. Martin 2011) was used to trim adapter sequences and low quality bases from the reads. Reads were aligned to the Chinese Hamster genome assembly PICR (101. Rupp et al. 2018) and annotation GCF_003668045.1, part of the NCBI Annotation Release 103. Sequence alignment was accomplished using the STAR v2.5.3a aligner (94. Dobin et al. 2013) with default parameters. Reads mapped to multiple locations were removed from analysis.

Identification and Quantification of Protein-coding TSSs: To call Transcription Start Site peaks, the Homer version 4.10 5′GRO-Seq pipeline was used (http://homer.ucsd.edu/homer/ngs/tss/index.html) (95. Duttke et al. 2019). Briefly, aligned reads for TSS samples and control samples were estimated to have a fragment size of 1 base pair (bp). Counts, or tags, were normalized to a million mapped reads, or counts per million (CPM). Regions of the genome were then scanned at a width of 150 bps and local regions with the maximum density of tags are considered clusters. Once initial clusters are called, adjacent, less dense regions 2× the peak width nearby are excluded to eliminate ‘piggyback peaks’ feeding off of signal from nearby large peaks. Those tags are redistributed to further regions and new clusters may be formed in this way. This process of cluster finding and nearby region exclusion continues until all tags are assigned to specific clusters. For all clusters, a tag threshold is established to filter out clusters occurring by random chance. These are modelled as a Poisson distribution to identify the expected number of tags. An FDR of 0.001 is used for multiple hypothesis correction. Importantly, in experiments where the cap is enriched, efficiency is not perfect, and additional reads tend to occur in high-expressing genes. To correct for this, we use control samples, GRO-Seq and csRNA-input for GRO-Cap and csRNA-seq, respectively. These experiments do not enrich for the 5′ cap, and thus will be found along the gene body. We enforce our peaks to be more than 2-fold enriched compared to the controls. Motifs were visualized using HOMERs compareMotifs.pl (97. Heinz et al. 2010). Sample peaks were merged using the mergePeaks command in Homer. Briefly, if samples have overlapping peaks, they are combined into one, where the start position is the minimum start position and the end is maximum end position. Additionally, when merging the samples' peak expression in the same tissue, the average CPM was used.

Promoter TSS calling and Gene TSS Quantification: TSSs were assigned based on the nearest gene and mRNA transcript listed in the NCBI Annotation 103, released using the PICR genome. To annotate protein-coding TSSs, a distance threshold from the original annotation was enforced. Ultimately, we used a distance of −1 kb to +1 kb from the initial reported TSS. Additionally, any intron peaks and peaks going in the reverse direction from the gene were filtered out. To associate TSS expression with the gene, the TSSs are grouped by their nearby gene, and the TSS with maximum average CPM is used.

Identifying silenced DNA Repair Genes: We looked for DNA repair genes that are silenced in CHO, but are more expressed in other Hamster tissues. We detected genes in which CHO was lower than the average tissue. To do this, we calculated the log 2 counts per million (CPM) fold change of CHO compared to the average other Chinese Hamster tissues and Bone-marrow derived macrophage cell lines. We took these low scoring values. Those associated with DNA damage repair are listed in Table 2.

TABLE 2
DNA Damage Repair Genes that are Significantly
Transcriptionally Down Regulated in CHO Cells

| | | Relative | |
|---|---|---|---|
| | | Expression | |
| Gene | | (Fold change of | |
| ID | Gene Name | hamster/CHO) | Ontology |
| MCM7 | DNA replication licensing factor MCM7 | 2.96 | DNA replication |
| PPP2R5A | protein phosphatase 2 regulatory subunit | 1.68 | Homology-directed |
| | B′alpha | | repair |
| PIAS4 | E3 SUMO-protein ligase PIAS4 | 1.85 | DNA damage sensing |
| PBRM1 | Protein polybromo-1 | 1.69 | Chromatin modification |
| PARP2 | Poly (ADP-ribose) polymerase 2 | 1.03 | Chromatin modification |

*These DNA repair genes are transcriptionally suppressed in CHO cells, as discovered using a combination of GRO-Seq and mStart-Seq, and thus serve as targets for activation of DNA repair capabilities. We report the fold increase in expression seen across hamster tissues

Double-Strand Break Repair Quantitation

GFP Expression Assay

The EJ5-GFP reporter plasmid [55] (addgene #44026) was linearized with XhoI and transfected into CHO-K1 and CHO-SEAP using electroporation (Neon, Thermo Fisher). Genomic integration of the construct in individual clones was selected for through combined puromycin and hygromycin-B treatment at previously determined LD90 doses and validated through PCR (F: agcctctgttccacatacact (SEQ ID NO:1; R: ccagccaccaccttctgata (SEQ ID NO:2)). To run the GFP expression assay, cells carrying the reporter system are transfected with a custom DSB-inducing plasmid expressing both Cas9 and two sgRNAs targeting the 5′ and 3′ end of the spacer separating the GFP coding frame from its β-actin promoter (FIG. 1). To generate this plasmid, the Cas9 expression plasmid pSpCas9(BB)-2A-miRFP670 (addgene #91854) was linearized with DrdI/KpnI and ligated with the dual sgRNA expression cassette from pX333 (addgene #94073) (amplified with F: acgacctacaccgaactgag (SEQ ID NO:11), R: aggtcatgtactgggcacaa (SEQ ID NO:12)). Impaired DSB repair is detected by positive GFP expression. Expression of miRFP670 (far-red fluorescence) from the same plasmid serves as a transfection control. Quantification of unrepaired DSBs is done by first filtering for live cells (SSH/FSC gating) and then relating the fraction of both far-red positive and GFP positive cells to the total fraction of far-red positive cells.

SNP Reversal

A Cas9-tracrRNA complex was assembled in-vitro with an sgRNA targeting a PAM in proximity (<15 bp) to the respective SNP and transfected into cells with an 80 bp ssDNA-donor oligo carrying the corrected (Chinese hamster) sequence, following standard protocols (Integrated DNA Technologies). 48h after transfection single-cell clones were seeded onto 96-well plates, and successful SNP reversal was verified through restriction enzyme digestion and Sanger sequencing.

cDNA Knock-In

Total cDNA was prepared from primary Chinese hamster lung fibroblasts, and single cDNAs were amplified through RT-PCR following standard protocols (Invitrogen). cDNAs were cloned into a lentiviral backbone (pLJM1, addgene #91980) and transfected into HEK293T cells to generate lentiviral particles for transduction. Successful integration was screened for using antibiotic selection, and single cell clones were isolated from 96-well plates.

Fluorescence-Activated Cell Sorting (FACS)

Fluorescent protein expression is quantified on a FACS Canto II (BD) with 50,000 cells per sample. Appropriate gates for FSC, SSC, and far-red fluorescence are defined to select viable cells expressing the DSB inducer. Among these, gates are defined to relate GFP expressing cells to non-GFP expressing cells. Cell-sorting during the cDNA library knock-in screen is carried out on a BD Aria II Cell Sorter with the same gate settings to separate GFP-positive from GFP-negative cells. After sorting, recovered cells are cultivated for 2 days before lysis and extraction of genomic DNA (DNeasy, Qiagen).

TABLE 3
(Also referred to as Appendix 1), list of
DNA repair genes and mutations for repair.

| Gene ID | Gene Name | Variant |
|---|---|---|
| Rad1 | RAD1 | E125G |
| Tp53 | p53 | T211K |
| Prkdc | Protein kinase DNA-activated catalytic subunit | D1641N |
| Atm | Ataxia telangiectasia mutated | R2830H |
| Fancm | Fanconi anemia group M | E1432G |
| Mdm2 | transformed mouse 3T3 cell double minute 2 | E114G |
| Pttg1 | pituitary tumor-transforming 1 (“Securin”) | T91I |
| Wrn | Werner Syndrome helicase | V1096A |
| Prkdc | Protein kinase DNA-activated catalytic subunit | S3419G |
| Wrn | Werner Syndrome helicase | R879Q |
| Uvssa | UV stimulated scaffold protein A | T471M |
| Cdc20b | cell division cycle 20B | T230M |
| Clspn | Claspin | E651_E652del |
| Ccno | Cyclin O | T369M |
| Fancm | Fanconi anemia group M | N1758S |
| Polm | Polymerase Mu | A29S |
| Hltf | helicase like transcription factor | L328Q |
| Cdc20b | cell division cycle 20B | K255E |
| Neil1 | nei like DNA glycosylase 1 | E312D |
| Fancm | Fanconi anemia group M | E846D |
| Polq | Polymerase Theta | R929K |
| Xrcc1 | X-ray repair cross complementing 1 | R208L |
| Fancm | Fanconi anemia group M | T634M |
| Fanca | Fanconi anemia group A | I930V |
| Xrcc1 | X-ray repair cross complementing 1 | R376P |
| Chaf1a | Chromatin assembly factor 1a | P29A |
| Cdc25b | cell division cycle 25B | P183L |
| Rad21 | RAD21 | Q436del |
| Fanca | Fanconi anemia group A | R1368G |
| Xrcc1 | X-ray repair cross complementing 1 | S206P |
| Xrcc1 | X-ray repair cross complementing 1 | G459R |
| Cdc20b | cell division cycle 20B | R291W |
| Pttg1 | pituitary tumor-transforming 1 (“Securin”) | V7I |
| Fancd2 | Fanconi anemia group D2 | I344L |
| Tdp2 | tyrosyl-DNA phosphodiesterase 2 | G67R |
| Fanca | Fanconi anemia group A | F11V |
| Fanca | Fanconi anemia group A | T1372P |
| E2f2 | E2F transcription factor 2 | V170E |
| Cdc20b | cell division cycle 20B | Y351F |
| E2f2 | E2F transcription factor 2 | H161N |
| Ccno | Cyclin O | I23V |
| E2f2 | E2F transcription factor 2 | H161Q |
| E2f2 | E2F transcription factor 2 | S154F |
| E2f2 | E2F transcription factor 2 | E160K |
| Rfc5 | Replication factor C subunit 5 | S29delinsCSLLPATT |
| E2f2 | E2F transcription factor 2 | I159del |
| E2f2 | E2F transcription factor 2 | D26H |
| Chaf1a | Chromatin assembly factor 1a | P31T |
| Ccne1 | Cyclin E1 | G295R |
| Ercc3 | ERCC excision repair 5 | G31E |
| Zbtb17 | Zinc finger and BTB domain containing 17 | H471Y |
| Rbl1 | RB transcriptional corepressor like 1 | A36_A47dup |
| Rmnd5a | Required for meiotic nuclear division 5 homolog A | S85R |
| Ccnh | cyclin H | D193N |
| Lig3 | DNA Ligase 3 | I158F |
| Pif1 | PIF1 5′-to-3′ DNA helicase | P136delinsRLKLA |
| Ccnk | Cyclin K | P343S |
| Rmnd5a | Required for meiotic nuclear division 5 homolog A | V86D |
| Cetn2 | Centrin-2 | G37E |
| Tp53 | p53 | Y220C |
| Dclre1a | DNA cross-link repair 1A | F542V |
| Xrcc3 | X-ray repair cross complementing 3 | H56L |
| Palb2 | Partner and localizer of BRCA2 | T3971 |
| Tert | telomerase reverse transcriptase | H766Y |
| Ddx11 | DEAD/H-box helicase 11 | A614E |
| Dna2 | DNA replication helicase/nuclease 2 | P88A |
| Shprh | SNF2 histone linker PHD RING helicase | D1053E |
| Rfc5 | Replication factor C subunit 5 | T133S |
| Helq | Helicase POLQ-like | Y973H |
| Rif1 | Replication timing regulatory factor 1 | C1918W |
| Blm | Bloom Syndrome Protein | D1287N |
| Blm | Bloom Syndrome Protein | D973N |
| Polg | Polymerase Gamma | V811M |
| Palb2 | Partner and localizer of BRCA2 | D873E |
| Recql4 | ATP-dependent DNA helicase Q4 | E319K |
| Helq | Helicase POLQ-like | E270K |
| Rfc1 | Replication factor C subunit 1 | G645S |
| Rmi1 | RecQ mediated genome instability 1 | N261D |
| Xrcc6 | X-ray repair cross complementing 6 | Q606H |
| Espl1 | extra spindle pole bodies like 1 (“Separin”) | V1759M |
| Palb2 | Partner and localizer of BRCA2 | H57Y |
| Blm | Bloom Syndrome Protein | Y225C |
| Tert | telomerase reverse transcriptase | V274I |
| Pms1 | PMS1 | A162S |
| Rmi1 | RecQ mediated genome instability 1 | G476C |
| Recql4 | ATP-dependent DNA helicase Q4 | R769H |
| Ercc5 | ERCC excision repair 5 | N1179K |
| Rmi1 | RecQ mediated genome instability 1 | S291N |
| Cdc14b | cell division cycle 14B | L349F |
| Pnkp | Polynucleotide kinase 3′-phosphatase | I345V |
| Ercc5 | ERCC excision repair 5 | R1569G |
| Fancm | Fanconi anemia group M | V440I |
| Ppp2r5b | protein phosphatase 2 regulatory subunit B′beta | Q468K |
| Mpg | N-methylpurine DNA glycosylase | G5A |
| Brca2 | Breast cancer type 2 susceptibility protein | S2146F |
| Smc3 | structural maintenance of chromosomes 3 | R12P |
| Ccno | Cyclin O | S85A |
| Anapc2 | anaphase promoting complex subunit 2 | A21V |
| Anapc1 | anaphase promoting complex subunit 1 | V1620I |
| Ccno | Cyclin O | N82K |
| Dclre1b | DNA cross-link repair 1B | V353I |
| Dclre1a | DNA cross-link repair 1A | L227M |
| Rad23a | RAD23A | V156I |
| Parp2 | poly(ADP-ribose) polymerase 2 | E359K |
| Mbd4 | Methyl-CpG-binding domain protein 4 | P156S |
| Prpf19 | pre-mRNA processing factor 19 | S171N |
| Atm | Ataxia telangiectasia mutated | D1529N |
| E2f2 | E2F transcription factor 11 | S234G |
| Zbtb17 | Zinc finger and BTB domain containing 17 | I470_H471insY |
| Rad18 | RAD18 | S59F |
| Ccno | Cyclin O | C84G |
| Pkmyt1 | protein kinase membrane associated | R92Q |
| | tyrosine/threonine 1 | |
| Atm | Ataxia telangiectasia mutated | N2136H |
| E2f2 | E2F transcription factor 10 | L267F |
| Polq | Polymerase Theta | L75V |
| Msh3 | mutS homolog 3 | V908M |
| Dot1l | DOT1 like histone lysine methyltransferase | S377F |
| Ddb1 | damage specific DNA binding protein 1 | V866M |
| Fbxo18 | F-box DNA helicase 1 | K544R |
| Fbxo18 | F-box DNA helicase 1 | L71F |
| E2f2 | E2F transcription factor 9 | L233R |
| Polq | Polymerase Theta | E336D |
| Ccnd3 | Cyclin D3 | M82V |
| Brca2 | Breast cancer type 2 susceptibility protein | S142P |
| Brca2 | Breast cancer type 2 susceptibility protein | S43P |
| Lig4 | DNA Ligase 4 | D869N |
| Stag1 | Stromal antigen 1 | Q913R |
| Anapc5 | anaphase promoting complex subunit 5 | E98K |
| Ccnb3 | Cyclin B3 | K321N |
| Bub1b | BUB1 mitotic checkpoint serine/threonine kinase B | L123F |
| Fan1 | Fanconi-associated nuclease 1 | V793F |
| Ep300 | E1A binding protein p300 | G58D |
| Polg | Polymerase Gamma | D520N |
| Rfc1 | Replication factor C subunit 1 | A797P |
| Rfc1 | Replication factor C subunit 1 | A784P |
| E2f2 | E2F transcription factor 8 | S234delinsRPCRA |
| Smc6 | structural maintenance of chromosomes 6 | P538Q |
| Orc1 | origin recognition complex subunit 1 | S666P |
| Prkdc | Protein kinase DNA-activated catalytic subunit | G1421S |
| Ccnt1 | Cyclin T1 | P608L |
| Brip1 | Fanconi anemia group J | G396E |
| Xrcc2 | X-ray repair cross complementing 2 | H75Y |
| Polq | Polymerase Theta | L75H |
| Fancc | Fanconi anemia group C | L4S |
| Fancc | Fanconi anemia group C | L118S |
| Lig3 | DNA Ligase 3 | C759Y |
| Shprh | SNF2 histone linker PHD RING helicase | R347C |
| Helq | Helicase POLQ-like | G344E |
| Polq | Polymerase Theta | P2194S |
| Ung | Uracil-DNA glycosylase | G83E |
| Brsk2 | BR serine/threonine kinase 2 | R168C |
| Fancd2 | Fanconi anemia group D2 | P90L |
| Rad51b | RAD51 paralog B | G133R |
| Dclre1c | DNA cross-link repair 1c (Artemis) | H38L |
| Anapc11 | anaphase promoting complex subunit 11 | C33W |
| Atr | Ataxia telangiectasia and Rad3 related | P2147L |

| | Loss of | PROVEAN | # positive | cDNA | |
|---|---|---|---|---|---|
| Gene ID | Heterozygosity | Score | samples | Length | Ontology |
| Rad1 | yes | −6.383 | 11 | 1250 | DNA damage sensing |
| Tp53 | yes | −4.844 | 11 | 1836 | Cell cycle control |
| Prkdc | yes | −4.601 | 11 | 13099 | Non-homologous end-joining |
| Atm | yes | −4.455 | 11 | 12918 | DNA damage sensing |
| Fancm | yes | −4.334 | 11 | 6025 | Fanconi anemia |
| Mdm2 | yes | −3.698 | 11 | 2914 | Cell cycle control |
| Pttg1 | yes | −3.688 | 11 | 1162 | Chromosome segregation |
| Wrn | yes | −3.653 | 11 | 4749 | Helicases |
| Prkdc | yes | −2.964 | 11 | 13099 | Non-homologous end-joining |
| Wrn | yes | −2.478 | 11 | 4749 | Helicases |
| Uvssa | yes | −2.382 | 11 | 3188 | Nucleotide-excision repair |
| Cdc20b | yes | −2.108 | 11 | 1152 | Cell cycle control |
| Clspn | yes | −2.054 | 10 | 5108 | DNA damage sensing |
| Ccno | yes | −2.017 | 10 | 1164 | Cell cycle control |
| Fancm | yes | −1.994 | 11 | 6025 | Fanconi anemia |
| Polm | yes | −1.979 | 10 | 3330 | DNA replication |
| Hltf | yes | −1.976 | 11 | 3350 | DNA replication |
| Cdc20b | yes | −1.684 | 11 | 1152 | Cell cycle control |
| Neil1 | yes | −1.607 | 11 | 2279 | Base excision repair |
| Fancm | yes | −1.274 | 11 | 6025 | Fanconi anemia |
| Polq | yes | −1.18 | 11 | 8650 | DNA replication |
| Xrcc1 | yes | −1.145 | 11 | 1902 | single-strand break repair |
| Fancm | yes | −0.701 | 11 | 6025 | Fanconi anemia |
| Fanca | yes | −0.696 | 11 | 4398 | Fanconi anemia |
| Xrcc1 | yes | −0.605 | 11 | 1902 | single-strand break repair |
| Chaf1a | yes | −0.591 | 3 | 3198 | Chromatin modification |
| Cdc25b | yes | −0.567 | 11 | 3190 | Cell cycle control |
| Rad21 | yes | −0.498 | 8 | 2105 | Chromosome segregation |
| Fanca | yes | −0.465 | 11 | 4398 | Fanconi anemia |
| Xrcc1 | yes | −0.394 | 11 | 1902 | single-strand break repair |
| Xrcc1 | yes | −0.384 | 8 | 1902 | single-strand break repair |
| Cdc20b | yes | −0.38 | 11 | 1152 | Cell cycle control |
| Pttg1 | yes | −0.362 | 11 | 1162 | Chromosome segregation |
| Fancd2 | yes | −0.326 | 11 | 5780 | Fanconi anemia |
| Tdp2 | yes | −0.274 | 1 | 2002 | Non-homologous end-joining |
| Fanca | yes | −0.228 | 11 | 4398 | Fanconi anemia |
| Fanca | yes | −0.228 | 11 | 4398 | Fanconi anemia |
| E2f2 | yes | −0.188 | 4 | 4777 | Cell cycle control |
| Cdc20b | yes | −0.155 | 11 | 1152 | Cell cycle control |
| E2f2 | yes | −0.045 | 4 | 4777 | Cell cycle control |
| Ccno | yes | −0.042 | 10 | 1164 | Cell cycle control |
| E2f2 | yes | −0.041 | 4 | 4777 | Cell cycle control |
| E2f2 | yes | −0.041 | 4 | 4777 | Cell cycle control |
| E2f2 | yes | −0.014 | 4 | 4777 | Cell cycle control |
| Rfc5 | yes | −0.01 | 1 | 1418 | DNA replication |
| E2f2 | yes | −0.005 | 4 | 4777 | Cell cycle control |
| E2f2 | yes | −0.036 | 1 | 4777 | Cell cycle control |
| Chaf1a | yes | −0.048 | 2 | 3198 | Chromatin modification |
| Ccne1 | yes | −0.099 | 2 | 1811 | Cell cycle control |
| Ercc3 | yes | −0.374 | 1 | 2349 | Nucleotide-excision repair |
| Zbtb17 | yes | −0.72 | 1 | 2672 | Cell cycle control |
| Rbl1 | yes | −1.595 | 1 | 4923 | Cell cycle control |
| Rmnd5a | yes | −2.675 | 1 | 5444 | Cell cycle control |
| Ccnh | yes | −3.07 | 1 | 1209 | Cell cycle control |
| Lig3 | yes | −3.839 | 1 | 5826 | single-strand break repair |
| Pif1 | yes | −4.13 | 2 | 3441 | Helicases |
| Ccnk | yes | −4.494 | 1 | 2647 | Cell cycle control |
| Rmnd5a | yes | −6.498 | 1 | 5444 | Cell cycle control |
| Cetn2 | yes | −7.473 | 1 | 1139 | Chromosome segregation |
| Tp53 | yes | −8.821 | 1 | 1836 | Cell cycle control |
| Dclre1a | no (yes in DXB11) | −4.228 | 6 | 4231 | Fanconi anemia |
| Xrcc3 | no (yes in CHOK1- | −5.27 | 5 | 1564 | Homology-directed repair |
| | ECACC DNA) | | | | |
| Palb2 | no | −5.671 | 10 | 3717 | Homology-directed repair |
| Tert | no | −4.843 | 11 | 4456 | Telomere maintenance |
| Ddx11 | no | −4.478 | 11 | 3674 | DNA replication |
| Dna2 | no | −4.116 | 2 | 3595 | Helicases |
| Shprh | no | −3.703 | 2 | 6921 | DNA replication |
| Rfc5 | no | −3.552 | 10 | 1418 | DNA replication |
| Helq | no | −3.511 | 11 | 3738 | Fanconi anemia |
| Rif1 | no | −3.275 | 11 | 8736 | Non-homologous end-joining |
| Blm | no | −3.199 | 11 | 4555 | Helicases |
| Blm | no | −2.985 | 11 | 4555 | Helicases |
| Polg | no | −2.827 | 11 | 4666 | DNA replication |
| Palb2 | no | −2.703 | 10 | 3717 | Homology-directed repair |
| Recql4 | no | −2.659 | 11 | 4069 | Helicases |
| Helq | no | −2.585 | 10 | 3738 | Fanconi anemia |
| Rfc1 | no | −2.098 | 11 | 4756 | DNA replication |
| Rmi1 | no | −1.989 | 10 | 2994 | Homology-directed repair |
| Xrcc6 | no | −1.703 | 11 | 2107 | Non-homologous end-joining |
| Espl1 | no | −1.48 | 11 | 6613 | Chromosome segregation |
| Palb2 | no | −1.288 | 11 | 3717 | Homology-directed repair |
| Blm | no | −1.237 | 11 | 4555 | Helicases |
| Tert | no | −0.701 | 8 | 4456 | Telomere maintenance |
| Pms1 | no | −0.548 | 11 | 3081 | Mismatch repair |
| Rmi1 | no | −0.522 | 9 | 2994 | Homology-directed repair |
| Recql4 | no | −0.351 | 11 | 4069 | Helicases |
| Ercc5 | no | −0.325 | 11 | 5453 | Nucleotide-excision repair |
| Rmi1 | no | −0.278 | 10 | 2994 | Homology-directed repair |
| Cdc14b | no | −0.249 | 11 | 2604 | Cell cycle control |
| Pnkp | no | −0.2 | 9 | 1837 | Non-homologous end-joining |
| Ercc5 | no | −0.154 | 8 | 5453 | Nucleotide-excision repair |
| Fancm | no | −0.049 | 11 | 6025 | Fanconi anemia |
| Ppp2r5b | no | −0.045 | 8 | 2611 | Cell cycle control |
| Mpg | no | −0.061 | 1 | 1190 | Base excision repair |
| Brca2 | no | −0.072 | 7 | 10688 | Homology-directed repair |
| Smc3 | no | −0.09 | 4 | 4278 | Chromosome segregation |
| Ccno | no | −0.161 | 1 | 1164 | Cell cycle control |
| Anapc2 | no | −0.201 | 1 | 2706 | Cell cycle control |
| Anapc1 | no | −0.584 | 1 | 8302 | Cell cycle control |
| Ccno | no | −0.726 | 2 | 1164 | Cell cycle control |
| Dclre1b | no | −0.845 | 1 | 2712 | Fanconi anemia |
| Dclre1a | no | −0.871 | 5 | 4231 | Fanconi anemia |
| Rad23a | no | −0.98 | 1 | 1236 | Nucleotide-excision repair |
| Parp2 | no | −1.038 | 1 | 1852 | Chromatin modification |
| Mbd4 | no | −1.073 | 1 | 2566 | Base excision repair |
| Prpf19 | no | −1.285 | 1 | 2180 | DNA damage sensing |
| Atm | no | −1.364 | 1 | 12918 | DNA damage sensing |
| E2f2 | no | −1.371 | 1 | 4777 | Cell cycle control |
| Zbtb17 | no | −1.418 | 1 | 2672 | Cell cycle control |
| Rad18 | no | −1.552 | 1 | 2435 | DNA replication |
| Ccno | no | −1.633 | 1 | 1164 | Cell cycle control |
| Pkmyt1 | no | −2 | 1 | 2317 | Cell cycle control |
| Atm | no | −2.147 | 1 | 12918 | DNA damage sensing |
| E2f2 | no | −2.267 | 1 | 4777 | Cell cycle control |
| Polq | no | −2.272 | 7 | 8650 | DNA replication |
| Msh3 | no | −2.309 | 1 | 3994 | Mismatch repair |
| Dot1l | no | −2.322 | 1 | 6446 | Chromatin modification |
| Ddb1 | no | −2.332 | 1 | 4278 | Nucleotide-excision repair |
| Fbxo18 | no | −2.346 | 1 | 3397 | Helicases |
| Fbxo18 | no | −2.371 | 6 | 3397 | Helicases |
| E2f2 | no | −2.372 | 1 | 4777 | Cell cycle control |
| Polq | no | −2.72 | 1 | 8650 | DNA replication |
| Ccnd3 | no | −3.016 | 1 | 1977 | Cell cycle control |
| Brca2 | no | −3.089 | 1 | 10688 | Homology-directed repair |
| Brca2 | no | −3.089 | 1 | 10688 | Fanconi anemia |
| Lig4 | no | −3.104 | 4 | 3209 | Non-homologous end-joining |
| Stag1 | no | −3.239 | 1 | 4292 | Chromosome segregation |
| Anapc5 | no | −3.38 | 1 | 8302 | Cell cycle control |
| Ccnb3 | no | −3.433 | 5 | 4130 | Cell cycle control |
| Bub1b | no | −3.479 | 7 | 3628 | Cell cycle control |
| Fan1 | no | −4.061 | 1 | 3745 | Fanconi anemia |
| Ep300 | no | −4.283 | 1 | 8679 | Chromatin modification |
| Polg | no | −4.403 | 1 | 4666 | DNA replication |
| Rfc1 | no | −4.438 | 1 | 4756 | DNA replication |
| Rfc1 | no | −4.438 | 1 | 4756 | DNA replication |
| E2f2 | no | −4.446 | 1 | 4777 | Cell cycle control |
| Smc6 | no | −4.481 | 4 | 3748 | Chromosome segregation |
| Orc1 | no | −4.674 | 1 | 2894 | DNA replication |
| Prkdc | no | −4.723 | 1 | 13099 | Non-homologous end-joining |
| Ccnt1 | no | −4.743 | 1 | 2287 | Cell cycle control |
| Brip1 | no | −5.229 | 1 | 5592 | Fanconi anemia |
| Xrcc2 | no | −5.298 | 1 | 2716 | Homology-directed repair |
| Polq | no | −5.472 | 7 | 8650 | DNA replication |
| Fancc | no | −5.5 | 1 | 2514 | Fanconi anemia |
| Fancc | no | −5.609 | 1 | 2514 | Fanconi anemia |
| Lig3 | no | −5.917 | 1 | 5826 | single-strand break repair |
| Shprh | no | −6.654 | 1 | 6921 | DNA replication |
| Helq | no | −6.682 | 1 | 3738 | Fanconi anemia |
| Polq | no | −6.936 | 1 | 8650 | DNA replication |
| Ung | no | −6.957 | 1 | 1616 | Base excision repair |
| Brsk2 | no | −6.973 | 1 | 2214 | Cell cycle control |
| Fancd2 | no | −7.624 | 3 | 5780 | Fanconi anemia |
| Rad51b | no | −7.833 | 1 | 2341 | Homology-directed repair |
| Dclre1c | no | −8.296 | 1 | 2155 | Non-homologous end-joining |
| Anapc11 | no | −9.692 | 1 | 8302 | Cell cycle control |
| Atr | no | −10 | 1 | 8040 | DNA damage sensing |

| | | | CHOK1 protein | | | CHOS | | | DXB11 | K1_SF | pgsa |
|---|---|---|---|---|---|---|---|---|---|---|---|
| Gene ID | C0101_DNA | CHOK1_ECACC_DNA | free_DNA | CHOK1_ref_genome_DNA | CHOS_DNA | landscape_DNA | CHOZ_DNA | DG44_DNA | DNA seq | DNA | DNA |
| Rad1 | −6.383 | −6.383 | −6.383 | −6.383 | −6.383 | −6.383 | −6.383 | −6.383 | −6.383 | −6.383 | −6.383 |
| Tp53 | −4.844 | −4.844 | −4.844 | −4.844 | −4.844 | −4.844 | −4.844 | −4.844 | −4.844 | −4.844 | −4.844 |
| Prkdc | −4.601 | −4.601 | −4.601 | −4.601 | −4.601 | −4.601 | −4.601 | −4.601 | −4.601 | −4.601 | −4.601 |
| Atm | −4.455 | −4.455 | −4.455 | −4.455 | −4.455 | −4.455 | −4.455 | −4.455 | −4.455 | −4.455 | −4.455 |
| Fancm | −4.334 | −4.334 | −4.334 | −4.334 | −4.334 | −4.334 | −4.334 | −4.334 | −4.334 | −4.334 | −4.334 |
| Mdm2 | −3.698 | −3.698 | −3.698 | −3.698 | −3.698 | −3.698 | −3.698 | −3.698 | −3.698 | −3.698 | −3.698 |
| Pttg1 | −3.688 | −3.688 | −3.688 | −3.688 | −3.688 | −3.688 | −3.688 | −3.688 | −3.688 | −3.688 | −3.688 |
| Wrn | −3.653 | −3.653 | −3.653 | −3.653 | −3.653 | −3.653 | −3.653 | −3.653 | −3.653 | −3.653 | −3.653 |
| Prkdc | −2.964 | −2.964 | −2.964 | −2.964 | −2.964 | −2.964 | −2.964 | −2.964 | −2.964 | −2.964 | −2.964 |
| Wrn | −2.478 | −2.478 | −2.478 | −2.478 | −2.478 | −2.478 | −2.478 | −2.478 | −2.478 | −2.478 | −2.478 |
| Uvssa | −2.382 | −2.382 | −2.382 | −2.382 | −2.382 | −2.382 | −2.382 | −2.382 | −2.382 | −2.382 | −2.382 |
| Cdc20b | −2.108 | −2.108 | −2.108 | −2.108 | −2.108 | −2.108 | −2.108 | −2.108 | −2.108 | −2.108 | −2.108 |
| Clspn | −2.054 | −2.054 | −2.054 | −2.054 | −2.054 | −2.054 | −2.054 | −2.054 | | −2.054 | −2.054 |
| Ccno | −2.017 | | −2.017 | −2.017 | −2.017 | −2.017 | −2.017 | −2.017 | −2.017 | −2.017 | −2.017 |
| Fancm | −1.994 | −1.994 | −1.994 | −1.994 | −1.994 | −1.994 | −1.994 | −1.994 | −1.994 | −1.994 | −1.994 |
| Polm | −1.979 | −1.979 | −1.979 | −1.979 | −1.979 | | −1.979 | −1.979 | −1.979 | −1.979 | −1.979 |
| Hltf | −1.976 | −1.976 | −1.976 | −1.976 | −1.976 | −1.976 | −1.976 | −1.976 | −1.976 | −1.976 | −1.976 |
| Cdc20b | −1.684 | −1.684 | −1.684 | −1.684 | −1.684 | −1.684 | −1.684 | −1.684 | −1.684 | −1.684 | −1.684 |
| Neil1 | −1.607 | −1.607 | −1.607 | −1.607 | −1.607 | −1.607 | −1.607 | −1.607 | −1.607 | −1.607 | −1.607 |
| Fancm | −1.274 | −1.274 | −1.274 | −1.274 | −1.274 | −1.274 | −1.274 | −1.274 | −1.274 | −1.274 | −1.274 |
| Polq | −1.18 | −1.18 | −1.18 | −1.18 | −1.18 | −1.18 | −1.18 | −1.18 | −1.18 | −1.18 | −1.18 |
| Xrcc1 | −1.145 | −1.145 | −1.145 | −1.145 | −1.145 | −1.145 | −1.145 | −1.145 | −1.145 | 1.145 | −1.145 |
| Fancm | −0.701 | −0.701 | −0.701 | −0.701 | −0.701 | −0.701 | −0.701 | −0.701 | −0.701 | −0.701 | −0.701 |
| Fanca | −0.696 | −0.696 | −0.696 | −0.696 | −0.696 | −0.696 | −0.696 | −0.696 | 10.696 | −0.696 | −0.696 |
| Xrcc1 | −0.605 | −0.605 | −0.605 | −0.605 | −0.605 | −0.605 | −0.605 | −0.605 | −0.605 | −0.605 | −0.605 |
| Chaf1a | | | | | −0.591 | | −0.591 | | | | −0.591 |
| Cdc25b | −0.567 | −0.567 | −0.567 | −0.567 | −0.567 | −0.567 | −0.567 | −0.567 | −0.567 | −0.567 | −0.567 |
| Rad21 | −0.498 | −0.498 | | −0.498 | −0.498 | | −0.498 | | −0.498 | −0.498 | −0.498 |
| Fanca | −0.465 | −0.465 | −0.465 | −0.465 | −0.465 | −0.465 | −0.465 | −0.465 | −0.465 | 0.465 | −0.465 |
| Xrcc1 | −0.394 | −0.394 | −0.394 | −0.394 | −0.394 | −0.394 | −0.394 | −0.394 | −0.394 | −0.394 | −0.394 |
| Xrcc1 | −0.384 | | | −0.384 | −0.384 | −0.384 | −0.384 | −0.384 | −0.384 | | −0.384 |
| Cdc20b | −0.38 | −0.38 | −0.38 | −0.38 | −0.38 | −0.38 | −0.38 | −0.38 | −0.38 | −0.38 | −0.38 |
| Pttg1 | −0.362 | −0.362 | −0.362 | −0.362 | −0.362 | −0.362 | −0.362 | −0.362 | −0.362 | −0.362 | −0.362 |
| Fancd2 | −0.326 | −0.326 | −0.326 | −0.326 | −0.326 | −0.326 | −0.326 | −0.326 | −0.326 | −0.326 | −0.326 |
| Tdp2 | | | | | −0.274 | | | | | | |
| Fanca | −0.228 | −0.228 | −0.228 | −0.228 | −0.228 | −0.228 | −0.228 | −0.228 | −0.228 | −0.228 | −0.228 |
| Fanca | −0.228 | −0.228 | −0.228 | −0.228 | −0.228 | −0.228 | −0.228 | −0.228 | −0.228 | −0.228 | −0.228 |
| E2f2 | | | | −0.188 | −0.188 | −0.188 | −0.188 | | | | |
| Cdc20b | −0.155 | −0.155 | −0.155 | −0.155 | −0.155 | −0.155 | −0.155 | −0.155 | −0.155 | −0.155 | −0.155 |
| E2f2 | | | | −0.045 | −0.045 | −0.045 | −0.045 | | | | |
| Ccno | −0.042 | −0.042 | −0.042 | −0.042 | −0.042 | −0.042 | −0.042 | | −0.042 | −0.042 | −0.042 |
| E2f2 | | | | −0.041 | −0.041 | −0.041 | −0.041 | | | | |
| E2f2 | | | | −0.041 | −0.041 | −0.041 | −0.041 | | | | |
| E2f2 | | | | −0.014 | −0.014 | −0.014 | −0.014 | | | | |
| Rfc5 | | | | | −0.01 | | | | | | |
| E2f2 | | | | −0.005 | −0.005 | −0.005 | −0.005 | | | | |
| E2f2 | | | | | | | | | | | −0.036 |
| Chaf1a | | | −0.048 | −0.048 | | | | | | | |
| Ccne1 | −0.099 | | | −0.099 | | | | | | | |
| Ercc3 | | | | | | | | | | | −0.374 |
| Zbtb17 | | | | | | | | | | −0.72 | |
| Rbl1 | | | −1.595 | | | | | | | | |
| Rmnd5a | | | | | | | | −2.675 | | | |
| Ccnh | | | | | | | | | −3.07 | | |
| Lig3 | | | | | | | | −3.839 | | | |
| Pif1 | −4.13 | −4.13 | | | | | | | | | |
| Ccnk | | | | | | | | | −4.494 | | |
| Rmnd5a | | | | | | | | −6.498 | | | |
| Cetn2 | | | | | | | | | | | −7.473 |
| Tp53 | | | | | | | | | | | −8.821 |
| Dclre1a | | | −4.228 | −4.228 | | | −4.228 | | −4.228 | −4.228 | −4.228 |
| Xrcc3 | | −5.27 | | −5.27 | | | −5.27 | | | −5.27 | −5.27 |
| Palb2 | −5.671 | | −5.671 | −5.671 | −5.671 | −5.671 | −5.671 | −5.671 | −5.671 | −5.671 | −5.671 |
| Tert | −4.843 | −4.843 | −4.843 | −4.843 | −4.843 | −4.843 | −4.843 | −4.843 | −4.843 | −4.843 | −4.843 |
| Ddx11 | −4.478 | −4.478 | −4.478 | −4.478 | −4.478 | −4.478 | −4.478 | −4.478 | −4.478 | −4.478 | −4.478 |
| Dna2 | −4.116 | | | | −4.116 | | | | | | |
| Shprh | −3.703 | | | | −3.703 | | | | | | |
| Rfc5 | −3.552 | −3.552 | −3.552 | −3.552 | −3.552 | −3.552 | −3.552 | | −3.552 | −3.552 | −3.552 |
| Helq | −3.511 | −3.511 | −3.511 | −3.511 | −3.511 | −3.511 | −3.511 | −3.511 | −3.511 | −3.511 | −3.511 |
| Rif1 | −3.275 | −3.275 | −3.275 | −3.275 | −3.275 | −3.275 | −3.275 | −3.275 | −3.275 | −3.275 | −3.275 |
| Blm | −3.199 | −3.199 | −3.199 | −3.199 | −3.199 | −3.199 | −3.199 | −3.199 | −3.199 | −3.199 | −3.199 |
| Blm | −2.985 | −2.985 | −2.985 | −2.985 | −2.985 | −2.985 | −2.985 | −2.985 | −2.985 | −2.985 | −2.985 |
| Polg | −2.827 | −2.827 | −2.827 | −2.827 | −2.827 | −2.827 | −2.827 | −2.827 | −2.827 | −2.827 | −2.827 |
| Palb2 | −2.703 | −2.703 | −2.703 | −2.703 | −2.703 | | −2.703 | −2.703 | −2.703 | −2.703 | −2.703 |
| Recql4 | −2.659 | −2.659 | −2.659 | −2.659 | −2.659 | −2.659 | −2.659 | −2.659 | −2.659 | −2.659 | −2.659 |
| Helq | −2.585 | −2.585 | −2.585 | −2.585 | −2.585 | −2.585 | −2.585 | −2.585 | −2.585 | | −2.585 |
| Rfc1 | −2.098 | −2.098 | −2.098 | −2.098 | −2.098 | −2.098 | −2.098 | −2.098 | −2.098 | −2.098 | −2.098 |
| Rmi1 | −1.989 | −1.989 | −1.989 | −1.989 | −1.989 | | −1.989 | −1.989 | −1.989 | −1.989 | −1.989 |
| Xrcc6 | −1.703 | −1.703 | −1.703 | −1.703 | −1.703 | −1.703 | −1.703 | −1.703 | −1.703 | −1.703 | −1.703 |
| Espl1 | −1.48 | −1.48 | −1.48 | −1.48 | −1.48 | −1.48 | −1.48 | −1.48 | −1.48 | −1.48 | −1.48 |
| Palb2 | −1.288 | −1.288 | 1.288 | −1.288 | −1.288 | −1.288 | −1.288 | −1.288 | −1.288 | −1.288 | −1.288 |
| Blm | −1.237 | −1.237 | −1.237 | −1.237 | −1.237 | −1.237 | −1.237 | −1.237 | −1.237 | −1.237 | −1.237 |
| Tert | −0.701 | | | −0.701 | −0.701 | −0.701 | −0.701 | | −0.701 | −0.701 | −0.701 |
| Pms1 | −0.548 | −0.548 | −0.548 | −0.548 | −0.548 | −0.548 | −0.548 | −0.548 | −0.548 | −0.548 | −0.548 |
| Rmi1 | −0.522 | −0.522 | −0.522 | −0.522 | −0.522 | | −0.522 | | −0.522 | −0.522 | −0.522 |
| Recql4 | −0.351 | −0.351 | −0.351 | −0.351 | −0.351 | −0.351 | −0.351 | −0.351 | −0.351 | −0.351 | −0.351 |
| Ercc5 | −0.325 | −0.325 | −0.325 | −0.325 | −0.325 | −0.325 | −0.325 | −0.325 | −0.325 | −0.325 | −0.325 |
| Rmi1 | −0.278 | −0.278 | | −0.278 | −0.278 | −0.278 | −0.278 | −0.278 | −0.278 | −0.278 | −0.278 |
| Cdc14b | −0.249 | −0.249 | −0.249 | −0.249 | −0.249 | −0.249 | −0.249 | −0.249 | −0.249 | −0.249 | −0.249 |
| Pnkp | −0.2 | −0.2 | | −0.2 | −0.2 | −0.2 | −0.2 | | −0.2 | −0.2 | −0.2 |
| Ercc5 | −0.154 | −0.154 | | −0.154 | −0.154 | | −0.154 | | −0.154 | −0.154 | −0.154 |
| Fancm | −0.049 | −0.049 | −0.049 | −0.049 | −0.049 | −0.049 | −0.049 | −0.049 | −0.049 | −0.049 | −0.049 |
| Ppp2r5b | −0.045 | −0.045 | | −0.045 | −0.045 | | −0.045 | −0.045 | −0.045 | | −0.045 |
| Mpg | | | | | | | | −0.061 | | | |
| Brca2 | | −0.072 | −0.072 | −0.072 | | | −0.072 | | −0.072 | −0.072 | −0.072 |
| Smc3 | | | −0.09 | −0.09 | | | −0.09 | | | | −0.09 |
| Ccno | | | | −0.161 | | | | | | | |
| Anapc2 | | | | | | | | | −0.201 | | |
| Anapc1 | | | | | | | | | −0.584 | | |
| Ccno | −0.726 | | | −0.726 | | | | | | | |
| Dclre1b | | | | | | | | | | | −0.845 |
| Dclre1a | | −0.871 | −0.871 | −0.871 | | | −0.871 | | | | −0.871 |
| Rad23a | | | | | | | | | −0.98 | | |
| Parp2 | | | | | | | | | −1.038 | | |
| Mbd4 | | | | | | | | | | | −1.073 |
| Prpf19 | | | | | | | | | | | −1.285 |
| Atm | | | | | | | | | | | −1.364 |
| E2f2 | | | | −1.371 | | | | | | | |
| Zbtb17 | | | | | | | −1.418 | | | | |
| Rad18 | | | | | | | | | −1.552 | | |
| Ccno | | | | −1.633 | | | | | | | |
| Pkmyt1 | | | | | | | | | | | −2 |
| Atm | | | | | | | | −2.147 | | | |
| E2f2 | | | | | | | | | | | −2.267 |
| Polq | | −2.272 | −2.272 | −2.272 | | | −2.272 | | −2.272 | −2.272 | −2.272 |
| Msh3 | | | | | | | | | | | −2.309 |
| Dot1l | | | | | | | | | | | −2.322 |
| Ddb1 | | | | | | | | | −2.332 | | |
| Fbxo18 | | | | | | | | | −2.346 | | |
| Fbxo18 | | −2.371 | −2.371 | −2.371 | | | −2.371 | | −2.371 | | −2.371 |
| E2f2 | | | | −2.372 | | | | | | | |
| Polq | | | −2.72 | | | | | | | | |
| Ccnd3 | | | | | | | | | | | −3.016 |
| Brca2 | | | | −3.089 | | | | | | | |
| Brca2 | | | | −3.089 | | | | | | | |
| Lig4 | | −3.104 | | −3.104 | | | −3.104 | | | | −3.104 |
| Stag1 | | | | | | | | −3.239 | | | |
| Anapc5 | | | | | | | | | −3.38 | | |
| Ccnb3 | | | −3.433 | −3.433 | | | −3.433 | | −3.433 | −3.433 | |
| Bub1b | | −3.479 | −3.479 | −3.479 | | | −3.479 | | −3.479 | −3.479 | −3.479 |
| Fan1 | | | | | | | | | −4.061 | | |
| Ep300 | | | | | | | | | | | −4.283 |
| Polg | | | | | | | | | | | −4.403 |
| Rfc1 | | | | | | | | −4.438 | | | |
| Rfc1 | | | | | | | | −4.438 | | | |
| E2f2 | | | | −4.446 | | | | | | | |
| Smc6 | | | | −4.481 | | | −4.481 | | −4.481 | | −4.481 |
| Orc1 | | | | | | | | −4.674 | | | |
| Prkdc | | | | | | | | | | | −4.723 |
| Cont1 | | | | | | | | | | | −4.743 |
| Brip1 | | | | | | | | | −5.229 | | |
| Xrcc2 | | | | | | | | | −5.298 | | |
| Polq | | −5.472 | −5.472 | −5.472 | | | −5.472 | | −5.472 | −5.472 | −5.472 |
| Fancc | | | | | | | | | −5.5 | | |
| Fancc | | | | | | | | | −5.609 | | |
| Lig3 | | | | | | | | | −5.917 | | |
| Shprh | | | | | | | | | −6.654 | | |
| Helq | | | | | | | | | | | −6.682 |
| Polq | | | | | | | | | | | −6.936 |
| Ung | | | | | | | | | | | −6.957 |
| Brsk2 | | | | | | | | | | | −6.973 |
| Fancd2 | | −7.624 | −7.624 | | | | | | | −7.624 | |
| Rad51b | | | | | | | | | −7.833 | | |
| Dclre1c | | | | | | | | | −8.296 | | |
| Anapc11 | | | | −9.692 | | | | | | | |
| Atr | | | | | | | | | −10 | | |

### Example 2

Cell Culture and Cell Line Generation

CHO-K1 cells (ATCC: CCL-61) and CHO-SEAP cells [66] were cultured in F-12K medium (Gibco), or Iscove's Modified Dulbecco's Medium (IMDM), respectively, supplemented with 10% (v/v) fetal bovine serum (FBS, Corning) and 1% (v/v) penicillin/streptomycin (Gibco) at 37° C. under an atmosphere of 5% CO₂. Cells were passaged every 2-3 days. CHO-K1 EJ5-GFP and CHO-SEAP EJ5-GFP were generated by transfecting CHO-K1 cells, or CHO-SEAP cell respectively, with a XhoI-linearized EJ5-GFP plasmid (Addgene #44026) and subsequent combined selection with puromycin (7 μg/mL) and hygromycin (300 μg/mL). After two weeks of antibiotic selection, clonal populations were generated by seeding cells in limiting dilution on 96-well plates and visually selecting clonal colonies. EJ5-GFP insertion was verified by PCR (OneTaq, New England Biolabs). CHO-K1 ATM+ was generated by transfecting a clonal population of CHO-K1 EJ5-GFP with a Cas9:tracrRNA:sgRNA ribonucleotide particle (Integrated DNA Technologies), targeting R2830H in ATM (Gene ID: 100754226), and a homology donor oligo encoding the corrected sequence, following standard protocols (Integrated DNA Technologies). Clonal populations were generated through limiting dilution, and the R2830H site was screened by PCR for the presence of a TaqI site in the corrected locus and verified by Sanger sequencing (Eton Biosciences, San Diego). Sanger sequencing data was deconvoluted using the ICE Analysis Tool (Synthego). CHO-K1 ATM+ PRKDC+ was generated by transfecting a clonal population of CHO-K1 ATM+ with a Cas9:tracrRMA:sgRNA ribonucleotide particle, targeting D1641N in PRKDC (Gene ID: 100770748), and a homology donor oligo encoding the corrected sequence. Clonal populations were generated through limiting dilution, and the PRKDC D1641N site was screened by PCR for the presence of a BamHI site in the corrected locus and verified by Sanger sequencing. CHO-SEAP CMV::XRCC6 was generated by lentiviral integration of XRCC6 (Sequence ID: XM_007620460.2) into CHO-SEAP and subsequent two-week selection in puromycin (7 μg/mL), followed by transfection with XhoI-linearized EJ5-GFP, and selection with hygromycin (300 μg/mL). Tranfections were carried out using either a Neon electroporation system (ThermoFisher) (24-well format) or lipofection (Lipofectamine LTX, invitrogen) (12-well format), using the recommended protocols for CHO-K1. All cells were maintained under combined puromycin/hygromycin selection throughout the experiments to avoid loss of the EJ5-GFP insertion. ATM was inhibited with KU-60019 (Selleckchem).

Cloning of Chinese Hamster Genes and Lentiviral Transduction

Chinese Hamster (Cricetulus griseus) lung fibroblasts were a gift from George Yerganian. RNA extraction (RNeasy, Qiagen) and total cDNA synthesis (SuperScriptIII, Invitrogen) were carried out using standard protocols. cDNA was purified and concentrated using ethanol precipitation, and 1 μL purified total cDNA (100-200 ng) was was used to amplify target genes through high-fidelity PCR (Q5, New England Biolabs) with primers carrying restriction sites for subsequent cloning into pLJM1 (Addgene #19319) following standard protocols (New England Biolabs). For lentivirus generation, HEK293T cells (ATCC: CRL-1573) were transfected with a cocktail of 800 ng of psPAX2 packaging plasmid (Addgene #12260), 800 ng PMD2.g envelope plasmid (Addgene #12259), and 800 ng of pLJM1 carrying the target gene, in 6-well plates using standard protocols (Lipofectamine LTX, Invitrogen). 24h after transfection, wells were replaced with fresh DMEM medium (Gibco). After another 24h the virus-containing medium was harvested, spun (2000×g, 5 min) and filtered (0.45 μm) and added dropwise to CHO-SEAP acceptor cells with 8 μg/ml polybrene (Millipore Sigma).

EJ5-GFP Flow Cytometry Assays

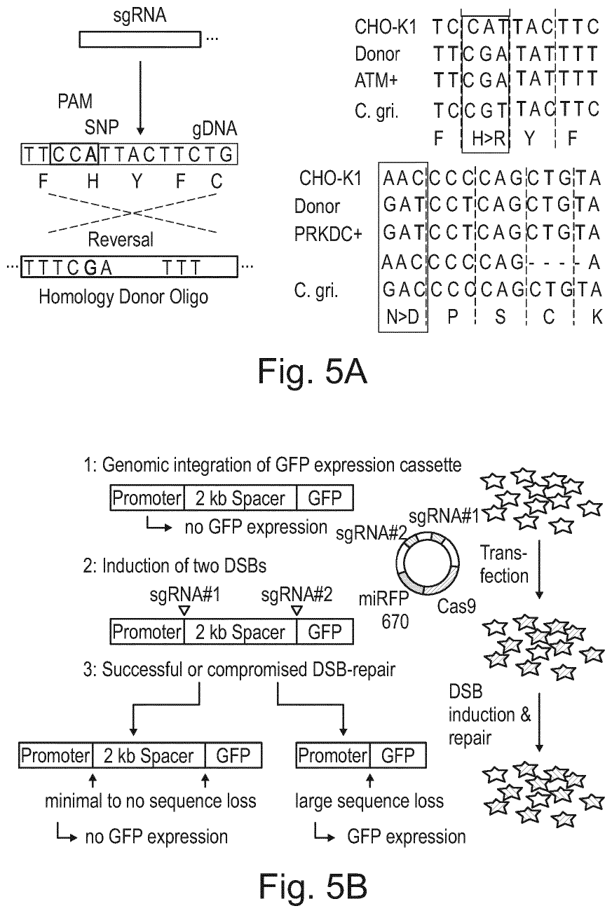

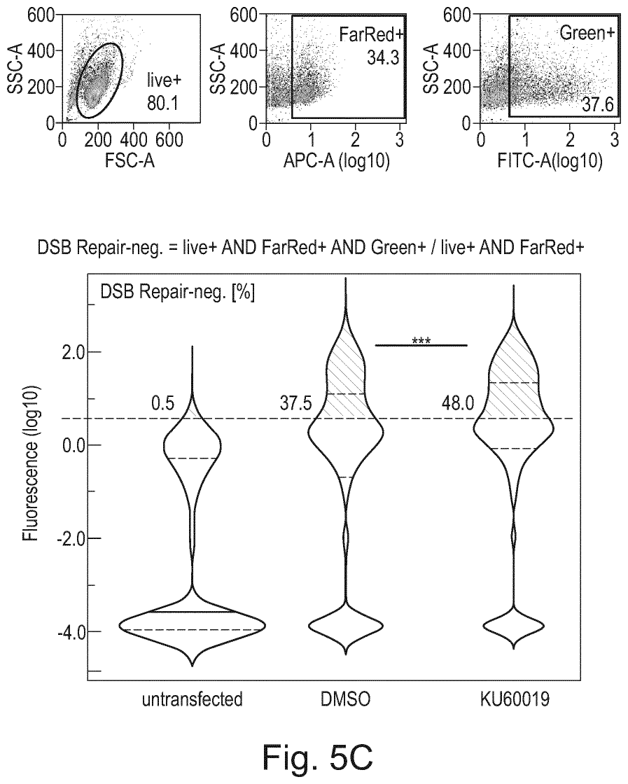

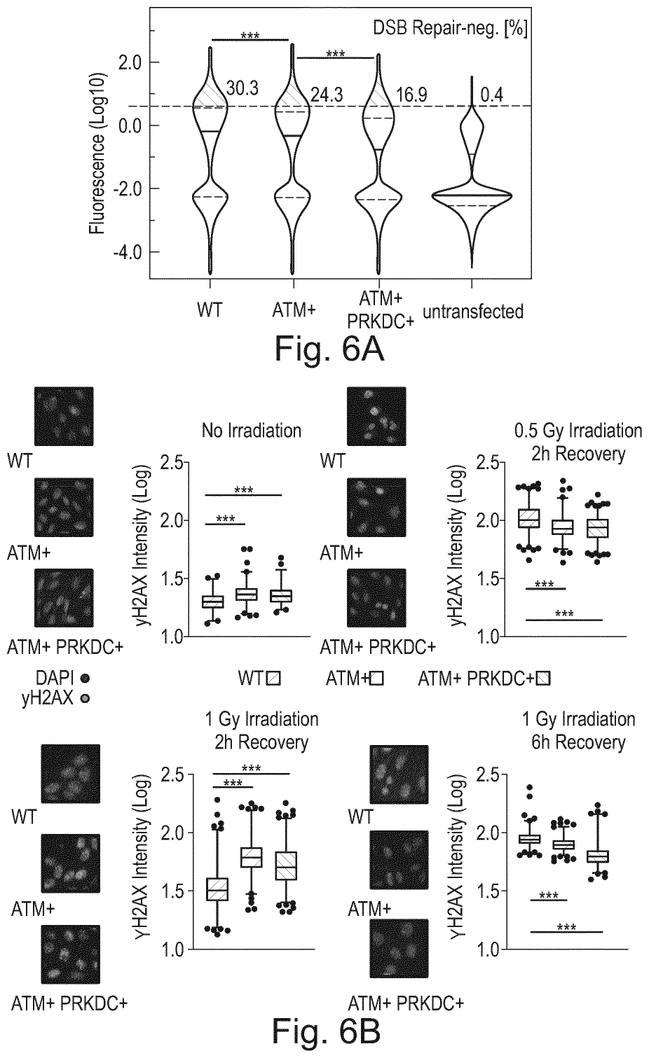

The DSB-inducer plasmid was constructed by ligation of two sgRNAs, targeting the EJ5-GFP cassette, into pX333 (Addgene #64073), and subsequent DrdI/KpnI-subcloning of the entire dual sgRNA expression cassette into pSpCas9(BB)-2A-miRFP670 (Addgene #91854). 30h after transfection of 1 μg of this plasmid (Lipofectamine LTX, Invitrogen; 12-well format), cells were trypsinized, resuspended in 250 μL DPBS (Gibco), and analyzed on a Canto II flow cytometer (BD Biosciences). Untransfected cells served as negative control to define proper gates in the APC and FITC channels for miRFP and GFP expression, respectively. DSB-repair negative cells were identified through boolean gating, as shown in FIG. 5c. Flow cytometry data was analyzed in FlowJo (BD Biosciences) and Prism (GraphPad).

Immunofluorescence, Comet Assays and Microscopy

Cells were seeded on chambered slides (Nunc, ThermoFisher) and, after attachment, either treated with the indicated doses of X-ray radiation (X-RSD 320, Precision X-ray), or incubated with 50 μg/mL bleocin (MilliporeSigma) for 1h. After the indicated recovery time, cells and fixated in 4% paraformaldehyde (ThermoFisher) for 10 min, washed in PBS (Gibco) for 2 min, and permeabilized with 0.5% Triton-X (Amresco) for 5 min, followed by washing for 5 min in PBS. After blocking with 5% goat-serum (MilliporeSigma) for 1h, cells were incubated in anti-γH2AX antibody (Cell Signaling Technology, Rabbit #9718) at 1:1000 dilution for 1h, washed three times in PBS-T (=0.1% Triton-X in PBS) for 5 min, and incubated with DyLight 488 goat-anti-rabbit (ThermoFisher) for 1h in the dark. After three washes in PBS-T for 5 min, cells were mounted in anti-fade mounting medium, containing DAPI (Vectashield Vibrance, Vector Laboratories). Samples were analyzed on a SP8 confocal microscope (Leica) with identical settings for gain and offset for each sample. Raw images were analyzed using custom MATLAB scripts (MathWorks), available on GitHub (https://github.com/PhilippSpahn/ImageProcessing). Briefly, individual nuclei were identified through segmentation of the DAPI channel, with manual adjustments in cases of touching or overlapping nuclei. Total γH2AX intensity was integrated per nucleus and normalized to nuclear size. Intensity integration was chosen instead of foci enumeration in order avoid problems with data intepretation in cases of indistinguishable separation of individual foci and to enable unbiased automated image processing. Comet assays were carried out following the manufacturer's protocol (Abcam), with 45 min electrophoresis at 1 V/cm in TBE-buffer. Slides were analzyed on a Axio Imager 2 (Zeiss) and processed using the OpenComet plug-in (www.cometbio.org/index.html) for ImageJ (NIH).

Karyotype Analysis

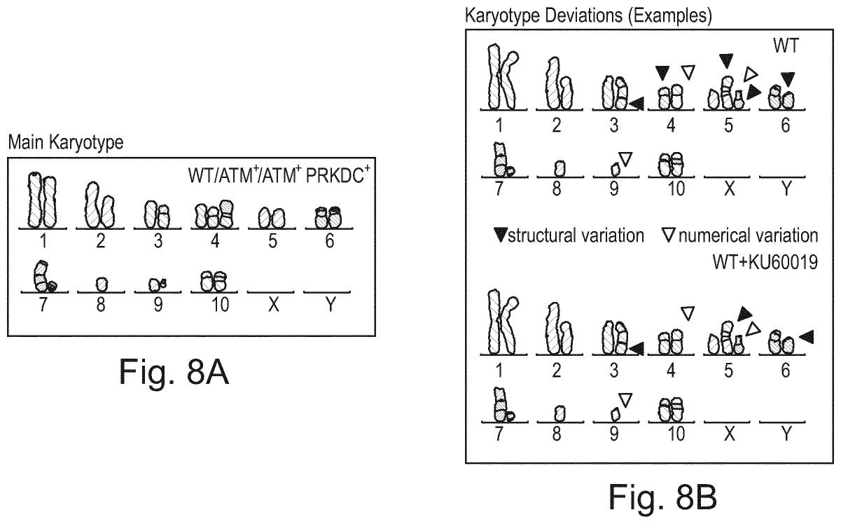

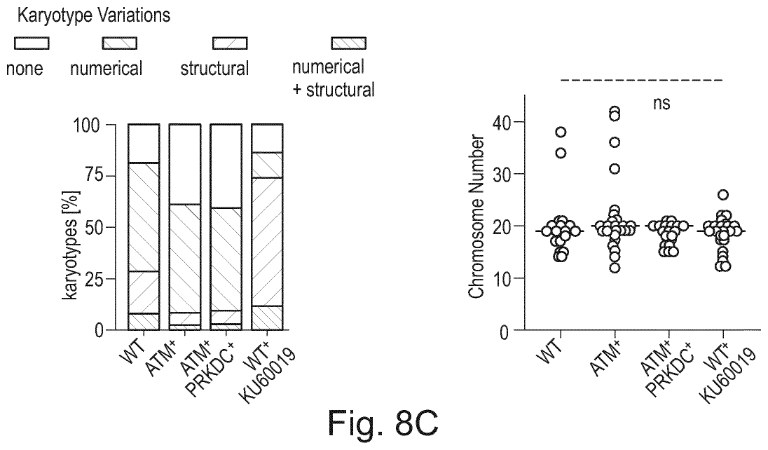

Metaphase spreads were prepared as previously described. Samples were labeled with multi-color DNA fluorescence in situ hybridization (FISH) probes (12× CHamster mFISH probe kit, MetaSystems) for spectral karyotyping as previously described [92]. For karyotypic analyses, the most abundant karyotype across samples was defined as the representative (“main”) karyotype, and deviations from this karyotype were scored as a numerical alteration (whole-chromosomal aneuploidy) and/or structural alteration (inter-chromosomal rearrangement, visible deletion). Structurally aberrant karyotypes (FIG. 8b) were defined as karyotypes showing at least one structural deviation from the representative karyotype.

Long-Term Culture

Cells were cultured in triplicates on 6-well plates. All cells were treated with 5 μM methotrexate (MTX) (MilliporeSigma) for 2 weeks at the beginning of the study (P0-P7) after which only one triplicate per genotype was continued under MTX until the rest of the study. Cells were cultured for 48 passages in total, with 3 passages/week. After. Protein titer was measured at P0, P7, and P48 using a SEAP reporter assay (Applied Biosystems, ThermoFisher).

DNA Oligos

Primers.

| | | |
|---|---|---|
| EJ5-GFP Insertion | F: AGCCTCTGTTCCACATACACT | SEQ ID NO: 1 |
| | R: CCAGCCACCACCTTCTGATA | SEQ ID NO: 2 |
| ATM R2830H | F: AGAGGTGTCCAGGCCAAGTT | SEQ ID NO: 3 |
| | R: GAGCTAACAATCAGCACGAACA | SEQ ID NO: 4 |
| PRKDC D1641N | F: AGAACCAGTTGCTGTAGTCTTGT | SEQ ID NO: 5 |
| | R: CCTGTGTGGTGATGGTGCATA | SEQ ID NO: 6 |
| CMV::XRCC6 | F: GCACCAAAATCAACGGGACT | SEQ ID NO: 7 |
| insertion | R: TCTTTCCCCTGCACTGTACC | SEQ ID NO: 8 |
| Cloning of C.gri. | F: TTATGCTAGCCCTTCTGTCCCTTTGGCTCG | SEQ ID NO: 9 |
| XRCC6 | R: TTATGAATTCTAAGTAGGTGGTCTGGCTGC | SEQ ID NO: 10 |
| Subcloning of | F: ACGACCTACACCGAACTGAG | SEQ ID NO: 11 |
| dual sgRNA | R: AGGTCATGTACTGGGCACAA | SEQ ID NO: 12 |
| expression locus | | |
| (px333) | | |

All primers were designed using Primer3 [93].

sgRNAs

| | | |
|---|---|---|
| Targetting | AGCCTCTGTTCCACATACACT | SEQ ID |
| ATMR2830H | | NO: 1 |
| Targetting | TGGCCAGGCTCTTACAGCTG | SEQ ID |
| PRKDC D1641N | | NO: 13 |
| DSB induction | AACAGGGTAATAATTCTACC | SEQ ID |
| (EJ5-GFP assay) | | NO: 14 |
| (5' end) | | |
| DSB induction | TAACAGGGTAATGGATCCAC | SEQ ID |
| (EJ5-GFP assay) | | NO: 15 |
| (3' end) | | |

ssDNA Oligos

| | | |
|---|---|---|
| ATM R2830H | GTTTCTCAAACCAAACAGCTGGGTCCAAGA | SEQ ID |
| homology | ATTTTTCCATACAAAAATATCGAAAAACTGG | NO: 16 |
| donor | TTCAAAGTTTTGGCAAATAGTCATGAAGGT | |
| | GTCA | |
| PRKDC D1641N | CATTGCTCCTGCAGAGGAAAGGCAGTGCCT | SEQ ID |
| homology | GCAATCATTGGATCCTAGCTGTAAGAGCCT | NO: 17 |
| donor | GGCCAATGGACTCCTGGAGTTAGCCT | |

SNP correction of DNA repair genes leads to an improved DNA damage response Through genome editing, we generated a clonal CHO-K1 population with a successful reversal of R2830H in ATM (hereafter referred to as CHO ATM+). In addition, from this population, we generated a sub-clone with a successful reversal of D1641N in PRKDC (hereafter referred to as CHO ATM+ PRKDC+) (FIG. 5a). These reversals were done in succession in the same cell line to assess the cumulative effect of DNA repair improvements. Whole transcriptome sequencing of the new cell lines ATM+ and ATM+ PRKDC+ revealed only few differentially expressed genes, and gene set enrichment analysis did not identify significantly up-/downregulated pathways, consistent with these SNP reversals not having detrimental effects on viability or metabolism.

To assess improvement in DSB repair capability in the ATM+ and ATM+ PRKDC+ cell lines, we implemented a GFP-based reporter system (based on the EJ5-GFP reporter [60]) that allows quantification of DSB repair through transient plasmid transfection and subsequent flow cytometry. This reporter is a gene expression cassette, comprising a GFP reading frame, separated from a constitutive promoter by a large (2 kb) spacer (FIG. 5b). Through transient transfection with a Cas9:miRFP plasmid expressing two sgRNAs targeting the 5′ and 3′ end of the spacer, two DSBs are generated whose inappropriate repair result in positive GFP signal providing a fast quantitative read-out of DSB repair ability (FIG. 5b). The assay was validated in CHO-K1 wildtype cells using KU-60019, a highly effective small-molecule inhibitor against ATM. Incubating cells with this inhibitor caused a significant increase in GFP+ positive cells, indicating compromised DSB repair (FIG. 5c). Since inhibition of ATM further exacerbated the DNA repair deficiency phenotype in cells carrying the ATM R2830H SNP, this mutation likely leads to only a hypomorphic allele in CHO-K1, rather than a full loss-of-function.

Running this assay on the novel, repair-optimized cell lines, CHO ATM+ showed a significant decrease in GFP signal, indicating a successful improvement in repair of the induced lesion (FIG. 6a). Even further improvement was seen in ATM+ PRKDC+(FIG. 6a). This indicates that DSB repair was successfully enhanced in these cell lines, and supports the notion that gradual restoration of DNA repair capability can be achieved by successive restoration of DNA repair genes carrying mutations in CHO.

To rule out effects potentially specific to the described GFP reporter, we analyzed DSB repair efficiency more generally, through immunostaining against γH2AX, a well-established cellular marker of DSBs. γH2AX denotes phosphorylated histone H2AX in the chromatin area surrounding a DSB which often extends several megabases from the break site, visible as a focus in confocal microscopy [61, 62]. Thus, quantification of γH2AX foci is often used as a read-out of unrepaired DSBs as H2AX is dephosphorylated only after repair has been initiated [63]. In CHO-K1, low levels of γH2AX foci are visible even in the absence of any DSB-generating treatments, corresponding to the endogenous origins of DSBs (FIG. 6b). It is important to note that the generation of γH2AX is partially dependent on the ATM kinase [64] which likely explains why under non-treated conditions foci intensity was slightly higher in the DNA-repair optimized CHO lines which carry a restored ATM gene and can thus likely mark damage sites more effectively. However, after a strong DSB-inducing treatment, ATM restoration should lead to a decrease in foci over time as breaks get repaired more efficiently. Indeed, after exposing cells to 1 Gy of X-ray radiation, foci intensity first increased more quickly in engineered cell lines, consistent with the improved damage sensing, but seen decreased faster over a recovery period of 6h, compared to wildtype cells (FIG. 6b). With lower doses of radiation, the faster decrease in foci intensity is visible after only a 2h recovery period (FIG. 6b). These observations confirm that the DSB repair machinery is more active in the engineered cell lines and shows improved response to ubiquitous DNA damage, not specific to a break triggered at a specific site.

Restoration of DNA Repair Improves Genome Stability in CHO-K1

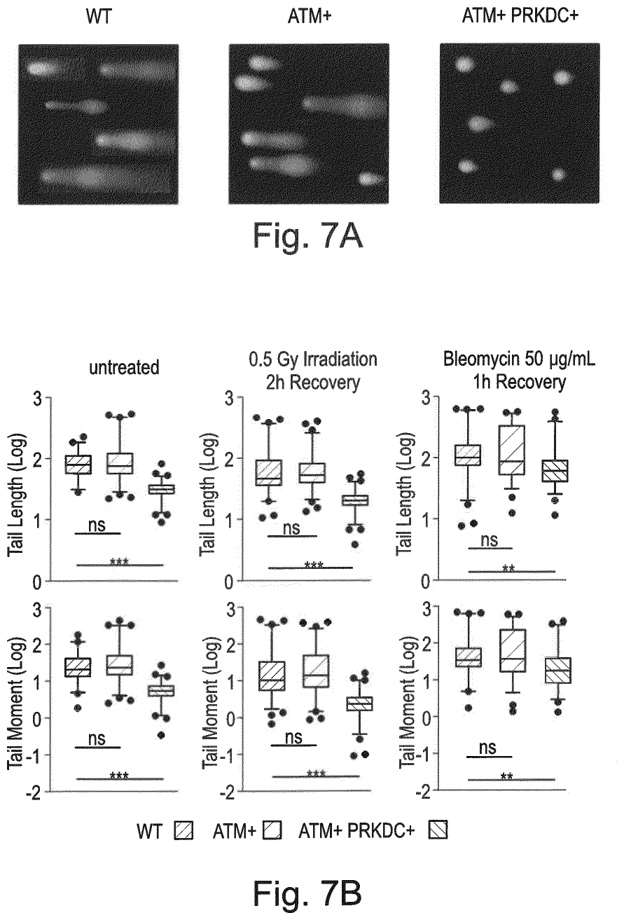

DSBs occur naturally in cell culture from endogenous metabolic processes or during DNA replication. If not repaired properly, a signal cascade through p53 stops the cell cycle until the damage is repaired [56]. p53 and other key cell cycle regulators carry likely deleterious SNPs in all CHO lines analyzed in this study. Thus, cell cycle control is likely dysfunctional which means that cell division continues despite persistent DSBs which can lead to chromosomal aberrations which ultimately drives transgene loss. We thus asked whether the improvements in the DNA damage response in the engineered CHO cell lines would improve the overall state of genome integrity. For this, we first exposed wildtype and engineered cell lines to DSB-inducing conditions and analyzed genome integrity on the single-cell level by electrophoresis where both the length and the intensity of the resulting DNA tail is an indicator of the amount of genome fragmentation (comet assay). After exposing cells to 0.5 Gy irradiation, followed by a 2h recovery period, we noticed longer DNA tails in wildtype CHO cells, with some cells exhibiting very long, bulky DNA tails indicating severe genome fragmentation due to persistent DSBs. Restoration of ATM did yield minor changes in DNA tail length, but additional restoration of PRKDC led to a strong reduction in both tail length and intensity, and we did not detect long bulky DNA tails in these samples (FIG. 7a). Similar results were obtained when exposing cells to high doses of the DSB-generating drug bleomycin (FIG. 7b). Together, these results indicate that restoration of two DNA repair genes enables significantly enhanced DNA repair and visibly reduces genome fragmentation. Importantly, even in the absence of genotoxic stress, we observed a certain degree of genome fragmentation (albeit at an overall lesser degree than under treatment) in wildtype CHO cell lines which was significantly ameliorated in our engineered cell lines (FIG. 7 b). This indicates that repair optimization not only improves genome integrity after artificial DSB induction but also under standard culture conditions.

Since unrepaired DSBs can lead to chromosomal aberrations, as mentioned above, we prepared karyotype samples of wildtype and engineered cell lines to analyze chromosomal aberrations on the single cell level. For this, both ATM+ and ATM+ PRKDC+ cell lines were cultured in parallel to the parental wildtype clone for a total of 60 passages (approx. 120 doublings) after which cells were arrested in mitosis, metaphase chromosomal spreads were prepared and stained with chromosome-specific probes (“chromosome painting”) to detect structural and numerical variations [65]. CHO karyotypes were previously shown to exhibit significant variation, regardless of culture supplementation or even clonal status. We also noticed considerable chromosome aberrations in karyotypes, such as major translocations, e.g. on chromosomes #3, #6, or #7, as well as whole chromosome duplications, e.g. #4 and loss of X-chromosomes (FIG. 8a). When we compared karyotypes across cell lines, we noticed a considerable reduction in structural aberrations in both engineered cell lines, evident as a significantly lower incidence of translocations and deletions (FIG. 8b), consistent with improved repair of DSBs and decreased genome fragmentation. A wild-type sample cultured under permanent supplementation with the ATM inhibitor KU-60019 served as a negative control and showed a massive increase in structural abnormalities (FIG. 8b). We did not see major stabilization with regard to chromosome number per karyotype among our cell lines (FIG. 8b), consistent with ATM and PRKDC having no direct role in chromosome segregation. Our dataset shows several likely deleterious SNPs in genes involved in chromosome segregation which would constitute interesting future targets to investigate chromosome number stability.

In summary, our data show that, while CHO cells carry a high burden in DNA repair genes, restoration of just few key genes leads to measurable improvements in DSBs repair, reduced genome fragmentation and an improvement in structural chromosomal stability.

Restoration of DNA Repair Improves Titer Stability in a Producing Cell Line

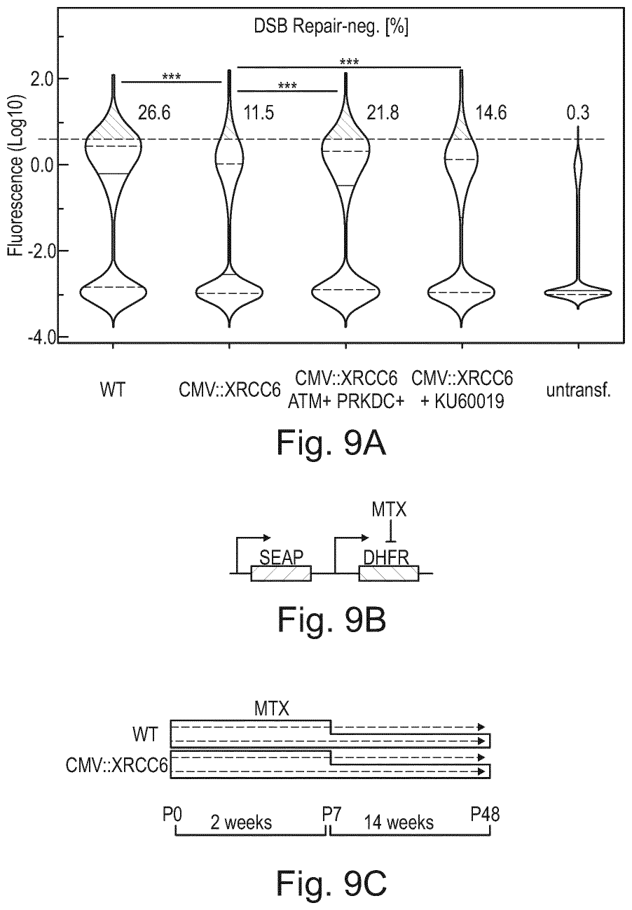

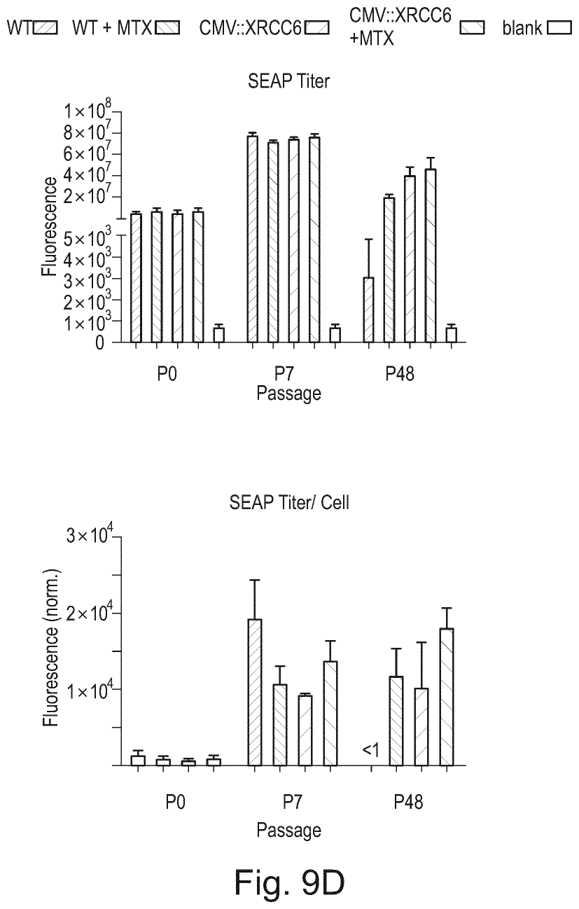

Genome instability often disrupts the maintenance of high protein titers in industrial biomanufacturing. Genome stabilization could counteract this problem by slowing the loss of transgene copies caused by chromosome instability. The results obtained in the CHO-K1 cell line presented above support the notion that engineering of DNA repair genes could help achieve this goal. Since CHO-K1 does not express any transgenes, we sought to apply this strategy in CHO-SEAP, an adherent cell line expressing human secreted alkaline phosphatase (SEAP) [66]. To explore additional gene targets from our SNP analysis, we selected XRCC6, another key component of the NHEJ repair pathway which carries a likely detrimental Q606H SNP in all 11 CHO lines in our dataset. We generated DNA repair-optimized CHO-SEAP cell line by expressing a Chinese Hamster wildtype copy of XRCC6 through lentiviral integration. The new cell line, CHO-SEAP CMV::XRCC6, showed significantly improved DSB repair, evident as a reduction of unsuccessful repair events by over 50% compared to CHO-SEAP wildtype in the EJ5-GFP assay (FIG. 9a). Surprisingly, reversals of the R2830H and D1641N SNPs in ATM and PRKDC, respectively, did not yield further improvements in this cell line, but instead caused a decrease in DSB-repair ability (FIG. 9a), opposite to what we observed in CHO-K1. Consistent with this observation, chemical inhibition of ATM resulted in improvement in repair ability (FIG. 9a), in contrast to our observations in CHO-K1 (see Discussion).

To finally investigate whether DNA-repair optimization has beneficial effects on transgene expression, we grew CHO-SEAP WT and CHO-SEAP CMV::XRCC6 alongside in a long-term culture experiment, and compared SEAP titer at the beginning and the end. Prior to the start of the experiment, cells were cultured in 5 uM methotrexate (MTX) for 1 week to select for high SEAP expression, after which MTX was taken off the growth medium in half of the samples (FIG. 9c). MTX is a competitive inhibitor of dihydrofolate reductase, an essential metabolic enzyme, which is co-expressed with the transgenic SEAP locus (FIG. 9b). While control cells grown under constant MTX supplementation showed no reduction in SEAP titer, wildtype cells grown without MTX showed a dramatic loss in SEAP titer by the end of the experiment. Interestingly, CMV::XRCC6 overexpression was sufficient to avoid this loss in titer, achieving comparable levels to MTX supplementation in the wildtype cell line (FIG. 9d). These results show that DNA repair optimization can lead to titer stabilization in a producing CHO cell line.

Faulty DNA repair has long been recognized as a major driver of genome instability [67-69]. Apart from few previous studies identifying impaired repair pathways [70, 71], this is the first report documenting the full extend of the mutational damage affecting DNA repair genes in various CHO cell lines. Moreover, while reactivation of silenced DNA repair genes has been successfully implemented before [72], restoration of DNA repair ability has not yet been systematically explored as a means to mitigate genome instability in the context of cell line development. This study is the first report to show that restoring DNA repair function through genome editing ameliorates genome stability in CHO. What is more, we show that despite the high mutational burden in DNA repair genes, restoration of just a single gene can yield measureable improvements in genome integrity. This makes DNA repair restoration a powerful and feasible novel addition to the cell line engineering toolbox. Our dataset of affected DNA repair genes opens up a plethora of options for future projects, targeting single genes or combinations of genes to develop novel cell lines for biopharmaceutical manufacturing with improved stability and productivity attributes. While effective alternative approaches have recently been described to increase productivity in CHO cells, such as overexpression of key metabolic genes [73], suppression of apoptosis [74], or design of novel promoters [75], restoration of DNA repair tackles the root mechanistic cause of genome instability and could thus enable long-lasting stability improvements. Beyond protein expression, restoration of DNA repair genes will likely prove effective in other aspects of cell line engineering, for example in the context of improving rates of targeted gene integration or gene correction in CHO [76]. Also, the approach could very likely be expanded to other mammalian cell lines.

As shown in this report, improvement of DSB repair ability appears to occur in an incremental fashion when combinations of DNA repair genes are being restored, provided these genes work synergistically. Finding such synergistic combinations is thus a main challenge. While literature data on human cancers, DNA repair, or evolutionary conservation [77] are a very helpful guide in hand-picking likely effective candidate genes, the unexpected results we obtained from ATM restoration and inhibition in CHO-SEAP are a warning sign. Given the divergent genomes of different CHO cell lines as well as the complex, intertwined nature of the mammalian DSB repair cascade [78], results from one cell line may not necessarily apply likewise to others. In mammals, DSB repair follows a “decision tree” [78] where pathway choice is largely determined by the severity of the DNA lesion. In particular, while a core NHEJ pathway can act independently of ATM [78, 79], ATM plays a key role in initiating repair of lesions requiring more pre-processing and more advanced repair pathways, such as homology-directed repair (HDR), alternative end-joining (aEJ), or the Fanconi anemia (FA) pathway [78, 80]. For this to be effective, genes in these pathways downstream of ATM need to be functional, and it is thus possible that in CHO-K1 these pathways have retained higher functionality that in CHO-SEAP. Indeed, our dataset shows a higher incidence of SNPs in HDR or FA pathways in CHO-SEAP (a DXB11 derivative) compared to CHO-K1. Thus, in CHO-SEAP ATM restoration might have triggered a negative net effect with downstream pathways being largely incapacitated, especially since the competition between pathways [81] could lead to inhibition of functional NHEJ. Previous studies have reported similar unexpected effects upon inhibition of key DNA repair genes, such as ATM or MRE11 [76, 82]. Observing opposite effects in different CHO cells after restoring identical genes thus provides a promising model platform to study synergistic gene relationships and competition within the DSB repair hierarchy.

Unlike ATM restoration, restoration of XRCC6 resulted in a considerable improvement in DSB repair, as indicated by the EJ5-GFP assay, although the SNP in XRCC6 is only heterozygous. Yet, Ku70 (the protein encoded by XRCC6) has to bind to Ku80 to form the heterodimeric Ku complex and mutations in XRCC6 are thus more likely to exert a dominant phenotype. Indeed, in human cells, a heterozygous Ku80 mutation is sufficient to trigger increased genome instability [83].

It is thus important to note that target choice needs to be carefully considered, and while data from the literature, heterozygosity status, or phenotype predictions can be helpful guides, prior testing or even screening of candidate genes is highly recommended. The EJ5-GFP cell ine described in this study can serve as an excellent discovery tool for this purpose. Certainly, this assay is approximate due to the possibility of false positive signal (i.e. a reporter site that didn't get cut despire the presense of Cas9:miRFP, or a reporter site whose lose ends failed to merge entirely), but it still provides a good estimate of DSB repair ability since positive GFP expression can only occur after imperfect DSB repair processing. In addition, we validated this assay using complementing DSB repair assessment methods. Thus, this built-in GFP reporter system is a useful technique that allows fast and efficient screening of even numerous candidate genes in.

To conclude, this study provides the first insight into the genetic basis of genome instability in CHO cells, and constitutes a proof-of-concept of the notion of DNA repair engineering as a powerful novel method for cell line development in industrial protein expression, and possibly beyond.

### REFERENCES

1. Walsh G (2018) Biopharmaceutical benchmarks 2018. Nature Biotechnology, 24(7):769-776. https://doi.org/10.1038/nbt.3040
2. Wang Q, Chung C Y, Chough S, Betenbaugh M J (2018) Antibody glycoengineering strategies in mammalian cells. Biotechnology and Bioengineering, 115(6):1378-1393. https://doi.org/10.1002/bit.26567
3. Dhara V G, Naik H M, Majewska N I, Betenbaugh M J (2018) Recombinant Antibody Production in CHO and NS0 Cells: Differences and Similarities. BioDrugs, 32(6):571-584. https://doi.org/10.1007/s40259-018-0319-9
4. Xu X, Nagarajan H, Lewis N E, Pan S, Cai Z, Liu X, Chen W, Xie M, Wang W, Hammond S, Andersen M R, Neff N, Passarelli B, Koh W, Fan H C, Wang J, Gui Y, Lee K H, Betenbaugh M J, Quake S R, Famili I, Palsson B O, Wang J (2011) The genomic sequence of the Chinese hamster ovary (CHO)-K1 cell line. Nature Biotechnology, 29(8):735-41. https://doi.org/10.1038/nbt.1932
5. Rupp O, MacDonald M L, Li S, Dhiman H, Polson S, Griep S, Heffner K, Hernandez I, Brinkrolf K, Jadhav V, Samoudi M, Hao H, Kingham B, Goesmann A, Betenbaugh M J, Lewis N E, Borth N, Lee K H (2018) A reference genome of the Chinese hamster based on a hybrid assembly strategy. Biotechnology and Bioengineering, 115(8):2087-2100. https://doi.org/10.1002/bit.26722
6. Lewis N E, Liu X, Li Y, Nagarajan H, Yerganian G, O'Brien E, Bordbar A, Roth A M, Rosenbloom J, Bian C, Xie M, Chen W, Li N, Baycin-Hizal D, Latif H, Forster J, Betenbaugh M J, Famili I, Xu X, Wang J, Palsson B O (2013) Genomic landscapes of Chinese hamster ovary cell lines as revealed by the Cricetulus griseus draft genome. Nature Biotechnology, 31(8):759-65. https://doi.org/10.1038/nbt.2624
7. Collins J H, Young E M (2018) Genetic engineering of host organisms for pharmaceutical synthesis. Current Opinion in Biotechnology, 53:191-200. https://doi.org/10.1016/j.copbio.2018.02.001
8. Ronda C, Pedersen L E, Hansen H G, Kallehauge T B, Betenbaugh M J, Nielsen A T, Kildegaard H F (2014) Accelerating genome editing in CHO cells using CRISPR Cas9 and CRISPy, a web-based target finding tool. Biotechnology and Bioengineering, 111(8):1604-1616. https://doi.org/10.1002/bit.25233
9. Lee J S, Grav L M, Lewis N E, Kildegaard H F (2015) CRISPR/Cas9-mediated genome engineering of CHO cell factories: Application and perspectives. Biotechnology Journal, 10(7):979-994. https://doi.org/10.1002/biot.201500082
10. Kildegaard H F, Baycin-Hizal D, Lewis N E, Betenbaugh M J (2013) The emerging CHO systems biology era: harnessing the 'omics revolution for biotechnology. Current Opinion in Biotechnology, 24(6):1102-7. https://doi.org/10.1016/j.copbio.2013.02.007
11. Stolfa G, Smonskey M T, Boniface R, Hachmann A B, Gulde P, Joshi A D, Pierce A P, Jacobia S J, Campbell A (2018) CHO-Omics Review: The Impact of Current and Emerging Technologies on Chinese Hamster Ovary Based Bioproduction. Biotechnology Journal, 13(3):1-14. https://doi.org/10.1002/biot.201700227
12. Daniotti J L, Vilcaes A a, Torres Demichelis V, Ruggiero F M, Rodriguez-Walker M (2013) Glycosylation of glycolipids in cancer: basis for development of novel therapeutic approaches. Frontiers in Oncology, 3(December):306. https://doi.org/10.3389/fonc.2013.00306
13. Kim J Y, Kim Y G, Lee G M (2012) CHO cells in biotechnology for production of recombinant proteins: Current state and further potential. Applied Microbiology and Biotechnology, 93(3):917-930. https://doi.org/10.1007/s00253-011-3758-5
14. Bailey L A, Hatton D, Field R, Dickson A J (2012) Determination of Chinese hamster ovary cell line stability and recombinant antibody expression during long-term culture. 50 Biotechnology and Bioengineering, 109(8):2093-2103. https://doi.org/10.1002/bit.24485
15. Fann C H, Guirgis F, Chen G, Lao M S, Piret J M (2000) Limitations to the amplification and stability of human tissue-type plasminogen activator expression by Chinese hamster ovary cells. Biotechnology and Bioengineering, 69(2):204-212. https://doi.org/10.1002/(SICI)1097-0290(20000720)69:2<204::AID-BIT9>3.0.CO;2-Z
16. Kim S J, Kim N S, Ryu C J, Hong H J, Lee G M (1998) Characterization of Chimeric Antibody Producing CHO Cells in the Course of Dihydrofolate Reductase-Mediated Gene Amplification and Their Stability in the Absence of Selective Pressure. Biotechnology and Bioengineering, 58(1)
17. Barnes L M, Bentley C M, Dickson A J (2003) Stability of protein production from recombinant mammalian cells. Biotechnology and Bioengineering, 81(6):631-639. https://doi.org/10.1002/bit.10517
18. Kim M, O'Callaghan P M, Droms K A, James D C (2011) A mechanistic understanding of production instability in CHO cell lines expressing recombinant monoclonal antibodies. Biotechnology and Bioengineering, 108(10):2434-2446. https://doi.org/10.1002/bit.23189
19. Beckmann T F, Krämer O, Klausing S, Heinrich C, Thüte T, B??ntemeyer H, Hoffrogge R, Noll T (2012) Effects of high passage cultivation on CHO cells: A global analysis. Applied Microbiology and Biotechnology, 94(3):659-671. https://doi.org/10.1007/s00253-011-3806-1
20. Veith N, Ziehr H, MacLeod R A F, Reamon-Buettner S M (2016) Mechanisms underlying epigenetic and transcriptional heterogeneity in Chinese hamster ovary (CHO) cell lines. BMC Biotechnology, 16(1):1-16. https://doi.org/10.1186/s12896-016-0238-0
21. Hammill L, Welles J, Carson G R (2000) The gel microdrop secretion assay: Identification of a low productivity subpopulation arising during the production of human antibody in CHO cells. Cytotechnology, 34(1-2):27-37. https://doi.org/10.1023/A:1008186113245
22. Baik J Y, Lee K H (2016) A framework to quantify karyotype variation associated with CHO production instability. Biotechnology and Bioengineering, 1-24. https://doi.org/10.1002/bit.26231
23. Dahodwala H, Lee K H (2019) The fickle CHO: a review of the causes, implications, and potential alleviation of the CHO cell line instability problem. Current Opinion in Biotechnology, 60(August 2018):128-137. https://doi.org/10.1016/j.copbio.2019.01.011
24. Chusainow J, Yang Y S, Yeo J H M, Ton P C, Asvadi P, Wong N S C, Yap M G S (2009) A study of monoclonal antibody-producing CHO cell lines: What makes a stable high producer?Biotechnology and Bioengineering, 102(4):1182-1196. https://doi.org/10.1002/bit.22158
25. Moritz B, Woltering L, Becker P B, Göpfert U (2016) High levels of histone H3 acetylation at the CMV promoter are predictive of stable expression in Chinese hamster ovary cells. Biotechnology Progress, 32(3):776-786. https://doi.org/10.1002/btpr.2271
26. Worton R G, Ho C C, Duff C (1977) Chromosome stability in CHO cells. Somatic cell genetics, 3(1):27-45. https://doi.org/10.1007/BF01550985
27. Cao Y, Kimura S, Itoi T, Honda K, Ohtake H, Omasa T (2012) Construction of BAC-based physical map and analysis of chromosome rearrangement in chinese hamster ovary cell lines. Biotechnology and Bioengineering, 109(6):1357-1367. https://doi.org/10.1002/bit.24347
28. Baik J Y, Lee K H (2017) Growth rate changes in CHO host cells are associated with karyotypic heterogeneity. Biotechnology Journal, 1-12.
29. Vcelar S, Jadhav V, Melcher M, Auer N, Hrdina A, Sagmeister R, Heffner K, Puklowski A, Betenbaugh M, Wenger T, Leisch F, Baumann M, Borth N (2018) Karyotype variation of CHO host cell lines over time in culture characterized by chromosome counting and chromosome painting. Biotechnology and Bioengineering, 115(1):165-173. https://doi.org/10.1002/bit.26453
30. Wurm F, Wurm M (2017) Cloning of CHO Cells, Productivity and Genetic Stability-A 50 Discussion. Processes, 5(2):20. https://doi.org/10.3390/pr5020020
31. Feichtinger J, Hernendez I, Fischer C, Hanscho M, Auer N, Hackl M, Jadhav V, Baumann M, Krempl P M, Schmidl C, Farlik M, Schuster M, Merkel A, Sommer A, Heath S, Rico D, Bock C, Thallinger G G, Borth N (2016) Comprehensive genome and epigenome characterization of CHO cells in response to evolutionary pressures and over time. Biotechnology and Bioengineering, 113(10):2241-2253. https://doi.org/10.1002/bit.25990
32. Richardson C, Moynahan M E, Jasin M (1998) Double-strand break repair by interchromosomal recombination: Suppression of chromosomal translocations. Genes and Development, 12(24):3831-3842. https://doi.org/10.1101/gad.12.24.3831
33. Gent D C Van, Hoeijmakers J H J, Kanaar R (2001) Chromosomal stability and the DNA double-stranded break connection. Nature Reviews Genetics, 2(3):196-206. https://doi.org/10.1038/35056049
34. Jackson SP (2002) Sensing and repairing DNA double-strand breaks. Carcinogenesis, 23(5):687-696. https://doi.org/10.1093/carcin/23.5.687
35. Ciccia A, Elledge S J (2010) The DNA Damage Response: Making It Safe to Play with Knives. Molecular Cell, 40(2):179-204. https://doi.org/10.1016/j.molcel.2010.09.019
36. Kaas C S, Kristensen C, Betenbaugh M J, Andersen M R (2015) Sequencing the CHO DXB11 genome reveals regional variations in genomic stability and haploidy. BMC Genomics, 16(1):1-9. https://doi.org/10.1186/s12864-015-1391-x
37. Lee J S, Kallehauge T B, Pedersen L E, Kildegaard H F (2015) Site-specific integration in CHO cells mediated by CRISPR/Cas9 and homology-directed DNA repair pathway. Scientific Reports, 1-11. https://doi.org/10.1038/srep08572
38. Pristovsek N, Nallapareddy S, Grav L M, Hefzi H, Lewis N E, Rugbjerg P, Hansen H G, Lee G M, Andersen M R, Kildegaard H F (2019) Systematic Evaluation of Site-Specific Recombinant Gene Expression for Programmable Mammalian Cell Engineering. ACS Synthetic Biology, 8(4):757-774. https://doi.org/10.1021/acssynbio.8b00453
39. Lee J S, Park J H, Ha T K, Samoudi M, Lewis N E, Palsson B O, Kildegaard H F, Lee G M (2018) Revealing Key Determinants of Clonal Variation in Transgene Expression in Recombinant CHO Cells Using Targeted Genome Editing. ACS Synthetic Biology, 7(12):2867-2878. https://doi.org/10.1021/acssynbio.8b00290
40. Gaidukov L, Wroblewska L, Teague B, Nelson T, Zhang X, Liu Y, Jagtap K, Mamo S, Allen Tseng W, Lowe A, Das J, Bandara K, Baijuraj S, Summers N M, Lu T K, Zhang L, Weiss R (2018) A multi-landing pad DNA integration platform for mammalian cell engineering. Nucleic Acids Research, 46(8):4072-4086. https://doi.org/10.1093/nar/gky216
41. Lee K H, Onitsuka M, Honda K, Ohtake H, Omasa T (2013) Rapid construction of transgene-amplified CHO cell lines by cell cycle checkpoint engineering. Applied Microbiology and Biotechnology, 97(13):5731-5741. https://doi.org/10.1007/s00253-013-4923-9
42. Matsuyama R, Yamano N, Kawamura N, Omasa T (2017) Lengthening of high-yield production levels of monoclonal antibody-producing Chinese hamster ovary cells by downregulation of breast cancer 1. Journal of Bioscience and Bioengineering, 123(3):382-389. https://doi.org/10.1016/j.jbiosc.2016.09.006
43. Khanna K K, Jackson S P (2001) DNA double-strand breaks: signaling, repair and the cancer connection. Nature Genetics, 27(3):247-54. https://doi.org/10.1038/85798
44. Bennardo N, Cheng A, Huang N, Stark J M (2008) Alternative-NHEJ is a mechanistically distinct pathway of mammalian chromosome break repair. PLoS Genetics, 4(6)https://doi.org/10.1371/journal.pgen.1000110
45. Hayduk E J, Lee K H (2005) Cytochalasin D can improve heterologous protein productivity in adherent Chinese hamster ovary cells. Biotechnology and Bioengineering, 90(3):354-364. https://doi.org/10.1002/bit.20438
46. Shiloh Y, Ziv Y (2013) The ATM protein kinase: regulating the cellular response to genotoxic stress, and more. Nature Reviews. Molecular Cell Biology, 14(4):197-210. https://doi.org/10.1038/nrm3546
47. Andrews S (2010) fastQC: A quality control tool for high throughput sequence data. https://www.bioinformatics.babraham.ac.uk/projects/fastqc/
48. Bolger A M, Lohse M, Usadel B (2014) Trimmomatic: A flexible trimmer for Illumina sequence data. Bioinformatics, 30(15):2114-2120. https://doi.org/10.1093/bioinformatics/btu170
49. Li H, Durbin R (2009) Fast and accurate short read alignment with Burrows-Wheeler transform. Bioinformatics, 25(14):1754-1760. https://doi.org/10.1093/bioinformatics/btp324
50. McKenna A, Hanna M, Banks E, DePristo M (2010) The Genome Analysis Toolkit: A MapReduce framework for analyzing next-generation DNA sequencing data. Genome Research, 20(1):1297-303. https://doi.org/10.1101/gr.107524.110.20
51. Cingolani P, Platts A, Wang L L, Lu X (2012) A program for annotating and predicting the effects of single nucleotide polymorphisms, SnpEff: SNPs in the genome of Drosophila melanogaster strain w1118; iso-2; iso-3. Fly, 6(2):1-13. https://doi.org/10.4161/fly.19695
52. Cingolani P, Patel V M, Coon M, Nguyen T, Land S J, Ruden D M, Lu X (2012) Using Drosophila melanogaster as a model for genotoxic chemical mutational studies with a new program, SnpSift. Frontiers in Genetics, 3(MAR):1-9. https://doi.org/10.3389/fgene.2012.00035
53. Wood R D, Mitchell M, Lindahl T (2005) Human DNA repair genes, 2005. Mutation Research—Fundamental and Molecular Mechanisms of Mutagenesis, 577(1-2 SPEC. ISS.):275-283. https://doi.org/10.1016/j.mrfmmm.2005.03.007
54. Choi Y, Sims G E, Murphy S, Miller J R, Chan A P (2012) Predicting the Functional Effect of Amino Acid Substitutions and Indels. PLoS ONE, 7(10)https://doi.org/10.1371/journal.pone.0046688
55. Bennardo N, Stark J M (2010) ATM limits incorrect end utilization during non-homologous end joining of multiple chromosome breaks. PLoS Genetics, 6(11):16-18. https://doi.org/10.1371/journal.pgen.1001194
56. Goodarzi A A, Jeggo P A (2013) The Repair and Signaling Responses to DNA Double-Strand Breaks. Advances in Genetics, 82https://doi.org/10.1016/B978-0-12-407676-1.00001-9
57. Goodwin J F, Knudsen K E (2014) Beyond DNA repair: DNA-PK function in cancer. Cancer Discovery, 4(10):1126-1139. https://doi.org/10.1158/2159-8290.CD-14-0358
58. Apostolou E, Stadtfeld M (2018) Cellular trajectories and molecular mechanisms of iPSC reprogramming. Current Opinion in Genetics and Development, 52:77-85. https://doi.org/10.1016/j.gde.2018.06.002
59. Mathieu A L, Verronese E, Rice G I, Fouyssac F, Bertrand Y, Picard C, Chansel M, Walter J E, Notarangelo L D, Butte M J, Nadeau K C, Csomos K, Chen D J, Chen K, Delgado A, Rigal C, Bardin C, Schuetz C, Moshous D, Reumaux H, Plenat F, Phan A, Zabot M T, Balme B, Viel S, Bienvenu J, Cochat P, Burg M Van Der, Caux C, Kemp E H, Rouvet I, Malcus C, Meritet J F, Lim A, Crow Y J, Fabien N, Menetrier-Caux C, Villartay J P De, Walzer T, Belot A (2015) PRKDC mutations associated with immunodeficiency, granuloma, and autoimmune regulator-dependent autoimmunity. Journal of Allergy and Clinical Immunology, 135(6):1578-1588.e5. https://doi.org/10.1016/j.jaci.2015.01.040
60. Bennardo N, Cheng A, Huang N, Stark J M (2008) Alternative-NHEJ is a mechanistically distinct pathway of mammalian chromosome break repair. PLoS Genetics, 4(6)https://doi.org/10.1371/journal.pgen.1000110
61. Rogakou E P, Boon C, Redon C, Bonner W M (1999) Megabase chromatin domains involved in DNA double-strand breaks in vivo. Journal of Cell Biology, 146(5):905-915. https://doi.org/10.1083/jcb.146.5.905
62. Podhorecka M, Skladanowski A, Bozko P (2010) H2AX Phosphorylation: Its Role in DNA Damage Response and Cancer Therapy. Journal of Nucleic Acids, 2010:1-9. https://doi.org/10.4061/2010/920161
63. Scarpato R, Castagna S, Aliotta R, Azzarb A, Ghetti F, Filomeni E, Giovannini C, Pirillo C, Testi S, Lombardi S, Tomei A (2013) Kinetics of nuclear phosphorylation (γ-H2AX) in human lymphocytes treated in vitro with UVB, bleomycin and mitomycin C. Mutagenesis, 28(4):465-473. https://doi.org/10.1093/mutage/get024
64. Paull T T (2015) Mechanisms of ATM Activation. Annual Review of Biochemistry, 84(1):711-738. https://doi.org/10.1146/annurev-biochem-060614-034335
65. Hu Q, Maurais E G, Ly P (2020) Cellular and genomic approaches for exploring structural chromosomal rearrangements. Chromosome Research, 19-30. https://doi.org/10.1007/s10577-020-09626-1
66. Hayduk E J, Lee K H (2005) Cytochalasin D can improve heterologous protein productivity in adherent Chinese hamster ovary cells. Biotechnology and Bioengineering, 90(3):354-364. https://doi.org/10.1002/bit.20438
67. Tubbs A, Nussenzweig A (2017) Endogenous DNA Damage as a Source of Genomic Instability in Cancer. Cell, 168:644-656. https://doi.org/10.1016/j.cell.2017.01.002
68. Jeggo P A, Pearl L H, Carr A M (2016) DNA repair, genome stability and cancer: a historical perspective. Nature Reviews. Cancer, 16(1):35-42. https://doi.org/10.1038/nrc.2015.4
69. Aguilera A, Garcia-Muse T (2013) Causes of genome instability. Annual Review of Genetics, 47:1-32. https://doi.org/10.1146/annurev-genet-111212-133232
70. Goth-Goldstein R (1980) Inability of Chinese Hamster Ovary Cells to Excise 06-Alkylguanine.

Cancer Research, 40(7):2623-2624.
71. Shen M R, Zdzienicka M Z, Mohrenweiser H, Thompson L H, Thelen M P (1998) Mutations in hamster single-strand break repair gene XRCC1 causing defective DNA repair. Nucleic Acids Research, 26(4):1032-1037.
72. Jeggo P A, Holliday R (1986) Azacytidine-induced reactivation of a DNA repair gene in Chinese hamster ovary cells. Molecular and Cellular Biology, 6(8):2944-2949. https://doi.org/10.1128/mcb.6.8.2944
73. Berger A, Fourn V Le, Masternak J, Regamey A, Bodenmann I, Girod P A, Mermod N (2020) Overexpression of transcription factor Foxa1 and target genes remediate therapeutic protein production bottlenecks in Chinese hamster ovary cells. Biotechnology and Bioengineering, 117(4):1101-1116. https://doi.org/10.1002/bit.27274
74. Xiong K, Marquart K F, Cour Karottki K J la, Li S, Shamie I, Lee J S, Gerling S, Yeo N C, Chavez A, Lee G M, Lewis N E, Kildegaard H F (2019) Reduced apoptosis in Chinese hamster ovary cells via optimized CRISPR interference. Biotechnology and Bioengineering, 116(7):1813-1819. https://doi.org/10.1002/bit.26969
75. Nguyen L N, Baumann M, Dhiman H, Marx N, Schmieder V, Hussein M, Eisenhut P, Hernandez I, Koehn J, Borth N (2019) Novel Promoters Derived from Chinese Hamster Ovary Cells via In Silico and In Vitro Analysis. Biotechnology Journal, 14(11)https://doi.org/10.1002/biot.201900125
76. Bosshard S, Duroy P O, Mermod N (2019) A role for alternative end-joining factors in homologous recombination and genome editing in Chinese hamster ovary cells. DNA Repair, 82(August):102691. https://doi.org/10.1016/j.dnarep.2019.102691
77. Brunette G J, Jamalruddin M A, Baldock R A, Clark N L, Bernstein K A (2019) Evolution-based screening enables genome-wide prioritization and discovery of DNA repair genes. Proceedings of the National Academy of Sciences, 116(39):201906559. https://doi.org/10.1073/pnas.1906559116
78. Scully R, Panday A, Elango R, Willis N A (2019) DNA double-strand break repair-pathway choice in somatic mammalian cells. Nature Reviews Molecular Cell Biology, 20(11):698-714. https://doi.org/10.1038/s41580-019-0152-0
79. Riballo E, KOhne M, Rief N, Doherty A, Smith G C M, Recio M J, Reis C, Dahm K, Fricke A, Krempler A, Parker A R, Jackson S P, Gennery A, Jeggo P A, Löbrich M (2004) A pathway of double-strand break rejoining dependent upon ATM, Artemis, and proteins locating to??-50 H2AX foci. Molecular Cell, 16(5):715-724. https://doi.org/10.1016/j.molcel.2004.10.029
80. Lim D, Kim S, Xu B, Maser RS (2000) ATM phosphorylates p95/nbs1 in an S-phase checkpoint pathway. Nature, 404(April):613-617.
81. Acid M, Pilla M, Perachon S, Sautel E, Mann A, Wermuth C G, Garrido F, Schwartz J, Everitt B J, Sokoloff P, Dyck E Van, Stasiak A Z, Stasiak A, West S C (1999) Binding of double-strand breaks in DNA by human Rad52 protein. Nature, 401(September):371-375.
82. Choi S, Gamper A M, White J S, Bakkenist C J (2010) Inhibition of ATM kinase activity does not phenocopy ATM protein disruption: Implications for the clinical utility of ATM kinase inhibitors. Cell Cycle, 9(20):4052-4057. https://doi.org/10.4161/cc.9.20.13471
83. Li G, Nelsen C, Hendrickson E A (2002) Ku86 is essential in human somatic cells. Proceedings of the National Academy of Sciences of the United States of America, 99(2):832-837. https://doi.org/10.1073/pnas.022649699
84. Bennardo N, Stark J M (2010) ATM limits incorrect end utilization during non-homologous end joining of multiple chromosome breaks. PLoS Genetics, 6(11):16-18. https://doi.org/10.1371/journal.pgen.1001194
85. Andrews S (2010) fastQC: A quality control tool for high throughput sequence data. https://www.bioinformatics.babraham.ac.uk/projects/fastqc/
86. Bolger A M, Lohse M, Usadel B (2014) Trimmomatic: A flexible trimmer for Illumina sequence data. Bioinformatics, 30(15):2114-2120. https://doi.org/10.1093/bioinformatics/btu170
87. Li H, Durbin R (2009) Fast and accurate short read alignment with Burrows-Wheeler transform. Bioinformatics, 25(14):1754-1760. https://doi.org/10.1093/bioinformatics/btp324
88. McKenna A, Hanna M, Banks E, DePristo M (2010) The Genome Analysis Toolkit: A MapReduce framework for analyzing next-generation DNA sequencing data. Genome Research, 20(1):1297-303. https://doi.org/10.1101/gr.107524.110.20
89. Cingolani P, Platts A, Wang L L, Lu X (2012) A program for annotating and predicting the effects of single nucleotide polymorphisms, SnpEff: SNPs in the genome of Drosophila melanogaster strain w1118; iso-2; iso-3. Fly, 6(2):1-13. https://doi.org/10.4161/fly.19695
90. Cingolani P, Patel V M, Coon M, Nguyen T, Land S J, Ruden D M, Lu X (2012) Using Drosophila melanogaster as a model for genotoxic chemical mutational studies with a new program, SnpSift. Frontiers in Genetics, 3(MAR):1-9. https://doi.org/10.3389/fgene.2012.00035
91. Szklarczyk D, Franceschini A, Wyder S, Forslund K, Heller D, Huerta-Cepas J, Simonovic M, Roth A, Santos A, Tsafou K P, Kuhn M, Bork P, Jensen U, Mering C von (2015) STRING v10: protein-protein interaction networks, integrated over the tree of life. Nucleic acids research, 43(Database issue):D447-52. https://doi.org/10.1093/nar/gku1003
92. Li H D, Lu C, Zhang H, Hu Q, Zhang J, Cuevas I C, Sahoo S S, Aguilar M, Maurais E G, Zhang S, Wang X, Akbay E A, Li G M, Li B, Koduru P, Ly P, Fu Y X, Castrillon D H (2020) A PoleP286R mouse model of endometrial cancer recapitulates high mutational burden and immunotherapy response. JCI insight, 5(14)https://doi.org/10.1172/jci.insight.138829
93. T K, M R (2007) Enhancements and modifications of primer design program. Bioinformatics, 23(10):1289-1291. https://doi.org/10.1093/bioinformatics/btm091
94. Dobin, Alexander, Carrie A. Davis, Felix Schlesinger, Jorg Drenkow, Chris Zaleski, Sonali Jha, Philippe Batut, Mark Chaisson, and Thomas R. Gingeras. 2013. “STAR: Ultrafast Universal RNA-Seq Aligner.” Bioinformatics 29 (1): 15-21.
95. Duttke, Sascha H., Max W. Chang, Sven Heinz, and Christopher Benner. 2019. “Identification and Dynamic Quantification of Regulatory Elements Using Total RNA.” Genome Research 29 (11): 1836-46.
96. Duttke, Sascha H. C., Scott A. Lacadie, Mahmoud M. Ibrahim, Christopher K. Glass, David L. Corcoran, Christopher Benner, Sven Heinz, James T. Kadonaga, and Uwe Ohler. 2015. “Human Promoters Are Intrinsically Directional.” Molecular Cell 57 (4): 674-84.
97. Heinz, Sven, Christopher Benner, Nathanael Spann, Eric Bertolino, Yin C. Lin, Peter Laslo, 50 Jason X. Cheng, Cornelis Murre, Harinder Singh, and Christopher K. Glass. 2010. “Simple Combinations of Lineage-Determining Transcription Factors Prime Cis-Regulatory Elements Required for Macrophage and B Cell Identities.” Molecular Cell 38 (4): 576-89.
98. Hetzel, Jonathan, Sascha H. Duttke, Christopher Benner, and Joanne Chory. 2016. “Nascent RNA Sequencing Reveals Distinct Features in Plant Transcription.” Proceedings of the National Academy of Sciences of the United States of America 113 (43): 12316-21.
99. Link, Verena M., Sascha H. Duttke, Hyun B. Chun, Inge R. Holtman, Emma Westin, Marten A. Hoeksema, Yohei Abe, et al. 2018. “Analysis of Genetically Diverse Macrophages Reveals Local and Domain-Wide Mechanisms That Control Transcription Factor Binding and Function.” Cell 173 (7): 1796-1809.e17.
100. Martin, Marcel. 2011. “Cutadapt Removes Adapter Sequences from High-Throughput Sequencing Reads.” EMBnet.journal. https://doi.org/10.14806/ej.17.1.200.
101. Rupp, Oliver, Madolyn L. MacDonald, Shangzhong Li, Heena Dhiman, Shawn Polson, Sven Griep, Kelley Heffner, et al. 2018. “A Reference Genome of the Chinese Hamster Based on a Hybrid Assembly Strategy.” Biotechnology and Bioengineering 115 (8): 2087-2100.

1. A method of preparing a cell for expression of a gene of interest, comprising reverting a mutation or a silencing of one or more DNA repair gene in the cell.

2. The method of claim 1, wherein the gene of interest has an increased expression level, compared to the expression in the unmodified cell.

3. The method of claim 1, wherein the cell has improved double strand break repair and/or genome stability, compared to the expression in the unmodified cell.

4. The method according to claim 1, wherein the cell has improved protein product titer, compared to the expression in the unmodified cell.

5. The method according to claim 1, wherein the one or more DNA repair gene targeted by reverting mutation are among the DNA repair machinery set forth in table 3.

6. The method according to claim 1, wherein the one or more DNA repair gene is selected from any one of XRCC6, ATM and/or PRKDC.

7. The method according to claim 1, wherein the one or more DNA repair gene is targeted for reversing a silencing.

8. The method according to claim 1, wherein the mutation includes SNPs and/or indels in CHO cells.

9. The method according to claim 1, wherein the one or more DNA repair gene has decreased expression in CHO cells, compared to native hamster tissue.

10. The method according to claim 1, which one or more DNA repair gene is one, at least two, at least three, at least four, at least five, at least six, at least 7, at least 8, at least 9, or at least 10 DNA repair genes.

11. The method according to claim 1, which cell is a CHO cell.

12. A cell made by the method of claim 1.

13. A method of producing a gene product comprising expressing a gene of interest in a cell made by the method of claim 1, and purifying the gene product.

14. A double-stranded break (DSB) reporter system providing quantitative detection of DSB repair efficiency in living cells.

15. The method according to claim 6, wherein the mutation is selected from any one of XRCC6 (Q606H), ATM (R2830H) and/or PRKDC (D1641 N).

16. The method according to claim 7, wherein the one or more DNA repair gene is selected from MCM7, PPP2R5A, PIAS4, PBRM1, and/or PARP2.

17. The method according to claim 11, wherein the CHO cell is selected from a CHO cell in table 1.

18. The method according to claim 17, wherein the CHO cell is selected from CHO-K1, CHO-K1/SF, CHO protein-free, CHO-DG44, CHO-S, C0101, CHO-Z, CHO-DXB11, and CHO-pgsA-745.


SEQUENCE LISTING (17 sequences)

SEQ ID NO: 1 - DNA, 21 bp, Artificial Sequence
  Artificial DNA primer
agcctctgttccacatacact

SEQ ID NO: 2 - DNA, 20 bp, Artificial Sequence
  Artificial DNA primer
ccagccaccaccttctgata

SEQ ID NO: 3 - DNA, 20 bp, Artificial Sequence
  Artificial DNA primer
agaggtgtccaggccaagtt

SEQ ID NO: 4 - DNA, 22 bp, Artificial Sequence
  Artificial DNA primer
gagctaacaatcagcacgaaca

SEQ ID NO: 5 - DNA, 23 bp, Artificial Sequence
  Artificial DNA primer
agaaccagttgctgtagtcttgt

SEQ ID NO: 6 - DNA, 21 bp, Artificial Sequence
  Artificial DNA primer
cctgtgtggtgatggtgcata

SEQ ID NO: 7 - DNA, 20 bp, Artificial Sequence
  Artificial DNA primer
gcaccaaaatcaacgggact

SEQ ID NO: 8 - DNA, 20 bp, Artificial Sequence
  Artificial DNA primer
tctttcccctgcactgtacc

SEQ ID NO: 9 - DNA, 30 bp, Artificial Sequence
  Artificial DNA primer
ttatgctagcccttctgtccctttggctcg

SEQ ID NO: 10 - DNA, 30 bp, Artificial Sequence
  Artificial DNA primer
ttatgaattctaagtaggtggtctggctgc

SEQ ID NO: 11 - DNA, 20 bp, Artificial Sequence
  Artificial DNA primer
acgacctacaccgaactgag

SEQ ID NO: 12 - DNA, 20 bp, Artificial Sequence
  Artificial DNA primer
aggtcatgtactgggcacaa

SEQ ID NO: 13 - DNA, 20 bp, Artificial Sequence
  Artificial DNA primer
tggccaggctcttacagctg

SEQ ID NO: 14 - DNA, 20 bp, Artificial Sequence
  Artificial DNA primer
aacagggtaataattctacc

SEQ ID NO: 15 - DNA, 20 bp, Artificial Sequence
  Artificial DNA primer
taacagggtaatggatccac

SEQ ID NO: 16 - DNA, 95 bp, Artificial Sequence
  Artificial ssDNA oligo
gtttctcaaaccaaacagctgggtccaagaatttttccatacaaaaatatcgaaaaactggttcaaagttttggcaaatagtcatgaaggtgtca

SEQ ID NO: 17 - DNA, 86 bp, Artificial Sequence
  Artificial ssDNA oligo
cattgctcctgcagaggaaaggcagtgcctgcaatcattggatcctagctgtaagagcctggccaatggactcctggagttagcct